SEQUENCE LISTING (24 sequences)

SEQ ID NO: 1 - protein, 645 aa, Xenotropic MuLV-related Virus VP35
MESPAFSKPLKDKINPWGPLIIMGILVRAGASVQRDSPHQVFNVTWKITNLMTGQTANATSLLGTMTDTFPKLYFDLCDLVGDNWDDPEPDIGDGCRSPGGRKRTRLYDFYVCPGHTVLTGCGGPREGYCGKWGCETTGQAYWKPSSSWDLISLKRGNTPKGQGPCFDSSVGSGSIQGATPGGRCNPLVLEFTDAGKRASWDAPKTWGLRLYRSTGADPVTLFSLTRQVLNVGPRVPIGPNPVITEQLPPSQPVQIMLPRPPRPPPSGAASMVPGAPPPSQQPGTGDRLLNLVEGAYQALNLTSPDKTQECWLCLVSGPPYYEGVAVLGTYSNHTSAPANCSVTSQHKLTLSEVTGQGLCIGAVPKTHQALCNTTQKTSDGSYYLASPAGTIWACSTGLTPCLSTTVLNLTTDYCVLVELWPKVTYHSPNYVYGQFGKKTKYKREPVSLTLALLLGGLTMGGIAAGVGTGTTALVATKQFEQLQAAIHTDLGALEKSVSALEKSLTSLSEVVLQNRRGLDLLFLKEGGLCAALKKECCFYADHTGVVRDSMAKLRERLNQRQKLFESGQGWFEGLFNRSPWFTTLISTIMGPLIVLLLILLFGPCILNRLVQFVKDRISVVQALVLTQQYHQLKSIDPEEVESRE

SEQ ID NO: 2 - protein, 1733 aa, Xenotropic MuLV-related Virus VP35
  misc_feature (537)..(537): Xaa can be any naturally occurring amino acid
MGQTVTTPLSLTLQHWGDVQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALTLSIKSKPPKPQVLPDSGGPLIDLLTEDPPPYGVQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEAGKAVRGNDGRPTQLPNEVNAAFPLERPDWDYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGDXGGQGQEPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVVGPMGQPLQVLTLNIENKYRLHETSKEPDVPLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSEQDCQRGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQAMLLDTDRVQFGPVVALNPATLLPLPEKEAPHDCLEILAETHGTRPDLTDQPIPDADYTWYTDGSSFLQEGQRRAGAAVTTETEVIWARALPAGTSAQRAELIALTQALKMAEGKKLNVYTDSRYAFATAHVHGEIYRRRGLLTSEGREIKNKNEILALLKALFLPKRLSIIHCPGHQKGNSAEARGNRMADQAAREAAMKAVLETSTLLIEDSTPYTPPHFHYTETDLKRLRELGATYNQTKGYWVLQGKPVMPDQSVFELLDSLHRLTHPSPQKMKALLDREESPYYMLNRDRTIQYVTETCTACAQVNASKAKIGAGVRVRGHRPGTHWEVDFTEVKPGLYGYKYLLVFVDTFSGWVEAFPTKRETAKVVTKKLLEDIFPRFGMPQVLGSDNGPAFASQVSQSVADLLGIDWKLHCAYRPQSSGQVERMNRTIKETLTKLTLASGTRDWVLLLPLALYRARNTPGPHGLTPYEILYGAPPPLVNFHDPEMSKLTNSPSLQAHLQALQAVQQEVWKPLAAAYQDQLDQPVIPHPFRVGDAVWVRRHQTKNLEPRWKGPYTVLLTTPTALKVDGISAWIHAAHVKAATTPPAGTAWKVQRSQNPLKIRLTRGAP

SEQ ID NO: 3 - protein, 536 aa, Xenotropic MuLV-related Virus VP35
MGQTVTTPLSLTLQHWGDVQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALTLSIKSKPPKPQVLPDSGGPLIDLLTEDPPPYGVQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEAGKAVRGNDGRPTQLPNEVNAAFPLERPDWDYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGD

SEQ ID NO: 4 - protein, 645 aa, Xenotropic MuLV-related Virus VP42
MESPAFSKPLKDKINPWGPLIIMGILVRAGASVQRDSPHQVFNVTWKITNLMTGQTANATSLLGTMTDTFPKLYFDLCDLVGDNWDDPEPDIGDGCRSPGGRKRTRLYDFYVCPGHTVLTGCGGPREGYCGKWGCETTGQAYWKPSSSWDLISLKRGNTPKGQGPCFDSSVGSGSIQGATPGGRCNPLVLEFTDAGKRASWDAPKTWGLRLYRSTGADPVTLFSLTRQVLNVGPRVPIGPNPVITEQLPPSQPVQIMLPRPPRPPPSGAASMVPGAPPPSQQPGTGDRLLNLVEGAYQALNLTSPDKTQECWLCLVSGPPYYEGVAVLGTYSNHTSAPANCSVTSQHKLTLSEVTGQGLCIGAVPKTHQALCNTTQKTSDGSYYLASPAGTIWACSTGLTPCLSTTVLNLTTDYCVLVELWPKVTYHSPNYVYGQFEKKTKYKREPVSLTLALLLGGLTMGGIAAGVGTGTTALVATKQFEQLQAAIHTDLGALEKSVSALEKSLTSLSEVVLQNRRGLDLLFLKEGGLCAALKEECCFYADHTGVVRDSMAKLRERLNQRQKLFESGQGWFEGLFNRSPWFTTLISTIMGPLIVLLLILLFGPCILNRLVQFVKDRISVVQALVLTQQYHQLKSIDPEEVESRE

SEQ ID NO: 5 - protein, 1733 aa, Xenotropic MuLV-related Virus VP42
  misc_feature (537)..(537): Xaa can be any naturally occurring amino acid
MGQTVTTPLSLTLQHWGDVQRIASNQSVDVKKRRWVTFCSAEWPTFNVGWPQDGTFNLGIISQVKSRVFCPGPHGHPDQVPYIVTWEALAYDPPPWVKPFVSPKPPPLPTAPVLPPGPSAQPPSRSALYPALTPSIKSKPPKPQVLPDSGGPLIDLLTEDPPPYGAQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEARKAVRGNDGRPTQLPNEVNAAFPLERPDWGYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGDXGGQGQEPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVVGPMGQPLQVLTLNIEDEYRLHETSKEPDVPLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSEQDCQRGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQAMLLDTDRVQFGPVVALNPATLLPLPEKEAPHDCLEILAETHGTRPDLTDQPIPDADYTWYTDGGSFLQEGQRRAGAAVTTETEVIWGGVLPAGTSAQRAELIALTQALKMAEGKKLNVYTDSRYAFATAHVHGEIYRRRGLLTSEGREIKNKNEILALLKALFLPKRLSIIHCPGHQKGNSAEARGNRMADQAAREAAMKAVLETSTLLIEDSTPYTPPHFHYTETDLKRLRELGATYNQTKGYWVLQGKPVMPDQSVFELLDSLHRLTHLSPQKMKALLDREESPYYMLNRDRTIQYVTETCTACAQVNASKAKIGAGVRVRGHRPGTHWEVDFTEVKPGLYGYKYLLVFVDTFSGWVEAFPTKRETAKVVSKKLLEDIFPRFGMPQVLGSDNGPAFASQVSQSVADLLGIDWKLHCAYRPQSSGQVERMNRTIKETLTKLTLASGTRDWVLLLPLALYRARNTPGPHGLTPYEILYGAPPPLVNFHDPEMSKLTNSPSLQAHLQALQAVQQEVWKPLAAAYQDQLDQPVIPHPFRVGDAVWVRRHQTKNLEPRWKGPYTVLLTTPTALKVDGISAWIHAAHVKAATTPPAGTAWKVQRSQNPLKIRLTRGAP

SEQ ID NO: 6 - protein, 536 aa, Xenotropic MuLV-related Virus VP42
MGQTVTTPLSLTLQHWGDVQRIASNQSVDVKKRRWVTFCSAEWPTFNVGWPQDGTFNLGIISQVKSRVFCPGPHGHPDQVPYIVTWEALAYDPPPWVKPFVSPKPPPLPTAPVLPPGPSAQPPSRSALYPALTPSIKSKPPKPQVLPDSGGPLIDLLTEDPPPYGAQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEARKAVRGNDGRPTQLPNEVNAAFPLERPDWGYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGD

SEQ ID NO: 7 - protein, 645 aa, Xenotropic MuLV-related Virus VP62
MESPAFSKPLKDKINPWGPLIIMGILVRAGASVQRDSPHQVFNVTWKITNLMTGQTANATSLLGTMTDTFPKLYFDLCDLVGDNWDDPEPDIGDGCRSPGGRKRTRLYDFYVCPGHTVLTGCGGPREGYCGKWGCETTGQAYWKPSSSWDLISLKRGNTPKGQGPCFDSSVGSGSIQGATPGGRCNPLVLEFTDAGKRASWDAPKTWGLRLYRSTGADPVTLFSLTRQVLNVGPRVPIGPNPVITEQLPPSQPVQIMLPRTPRPPPSGAASMVPGAPPPSQQPGTGDRLLNLVEGAYLALNLTSPDKTQECWLCLVSGPPYYEGVAVLGTYSNHTSAPANCSVTSQHKLTLSEVTGQGLCIGAVPKTHQALCNTTQKTSDGSYYLASPAGTIWACSTGLTPCLSTTVLNLTTDYCVLVELWPKVTYHSPNYVYGQFEKKTKYKREPVSLTLALLLGGLTMGGIAAGVGTGTTALVATKQFEQLQAAIHTDLGALEKSVSALEKSLTSLSEVVLQNRRGLDLLFLKEGGLCAALKEECCFYADHTGVVRDSMAKLRERLNQRQKLFESGQGWFEGLFNRSPWFTTLISTIMGPLIVLLLILLFGPCILNRLVQFVKDRISVVQALVLTQQYHQLKSIDPEEVESRE

SEQ ID NO: 8 - protein, 1733 aa, Xenotropic MuLV-related Virus VP62
  misc_feature (537)..(537): Xaa can be any naturally occurring amino acid
MGQTVTTPLSLTLQHWGDVQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALTPSIKSKPPKPQVLPDSGGPLIDLLTEDPPPYGAQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEARKAVRGNDGRPTQLPNEVNAAFPLERPDWDYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGDXGGQGQEPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVVGPMGQPLQVLTVNIEDEYWLHDTRKEPDVPLGSTWLSDFLQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSEQDCQRGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKNAGKLTMGQPLVILAPHAVEALVKQPPDRWLSNARMTHYQAMLLDTDRVQFGPVVALNPATLLPLPEKEAPHDCLEILAETHGTRPDLTDQPIPDADYTWYTDGSSFLQEGQRRAGAAVTTETEVIWARALPAGTSAQRAELIALTQALKMAEGKKLNVYTDSRYAFATAHVHGEIYRRRGLLTSEGREIKNKNEILALLKALFLPKRLSIIHCPGHQKGNSAEARGNRMADQAAREAAMKAVLETSTLLIEDSTPYTPPHFHYTETDLKRLRELGATYNQTKGYWVLQGKPVMPDQSVFELLDSLHRLTHLSPQKMKALLDREESPYYMLNRDRTIQYVTETCTACAQVNASKAKIGAGVRVRGHRPGTHWEVDFTEVKPGLYGYKYLLVFVDTFSGWVEAFPTKRETAKVVSKKLLEDIFPRFGMPQVLGSDNGPAFASQVSQSVADLLGIDWKLHCAYRPQSSGQVERMNRTIKETLTKLTLASGTRDWVLLLPLALYRARNTPGPHGLTPYEILYGAPPPLVNFHDPEMSKLTNSPSLQAHLQALQAVQQEVWKPLAAAYQDQLDQPVIPHPFRVGDAVWVRRHQTKNLEPRWKGPYTVLLTTPTALKVDGISAWIHAAHVKAATTPPAGTAWKVQRSQNPLKIRLTRGAP

SEQ ID NO: 9 - protein, 536 aa, Xenotropic MuLV-related Virus VP62
MGQTVTTPLSLTLQHWGDVQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALTPSIKSKPPKPQVLPDSGGPLIDLLTEDPPPYGAQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEARKAVRGNDGRPTQLPNEVNAAFPLERPDWDYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGD

SEQ ID NO: 10 - protein, 645 aa, Xenotropic MuLV-related Virus VP62
MESPAFSKPLKDKINPWGPLIIMGILVRAGASVQRDSPHQVFNVTWKITNLMTGQTANATSLLGTMTDTFPKLYFDLCDLVGDNWDDPEPDIGDGCRSPGGRKRTRLYDFYVCPGHTVLTGCGGPREGYCGKWGCETTGQAYWKPSSSWDLISLKRGNTPKGQGPCFDSSVGSGSIQGATPGGRCNPLVLEFTDAGKRASWDAPKTWGLRLYRSTGADPVTLFSLTRQVLNVGPRVPIGPNPVITEQLPPSQPVQIMLPRPPRPPPSGAASMVPGAPPPSQQPGTGDRLLNLVEGAYQALNLTSPDKTQECWLCLVSGPPYYEGVAVLGTYSNHTSAPANCSVTSQHKLTLSEVTGQGLCIGAVPKTHQALCNTTQKTSDGSYYLASPAGTIWACSTGLTPCLSTTVLNLTTDYCVLVELWPKVTYHSPNYVYGQFEKKTKYKREPVSLTLALLLGGLTMGGIAAGVGTGTTALVATKQFEQLQAAIHTDLGALEKSVSALEKSLTSLSEVVLQNRRGLDLLFLKEGGLCAALKEECCFYADHTGVVRDSMAKLRERLNQRQKLFESRQGWFEGLFNRSPWFTTLISTIMGPLIVLLLILLFGPCILNRLVQFVKDRISVVQALVLTQQYHQLKSIDPEEVESRE

SEQ ID NO: 11 - protein, 1733 aa, Xenotropic MuLV-related Virus VP62
  misc_feature (537)..(537): Xaa can be any naturally occurring amino acid
MGQTVTTPLSLTLQHWGDVQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALTPSIKSKPPKPQVLPDSGGPLIDLLTEDPPPYGAQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEARKAVRGNDGRPTQLPNEVNAAFPLERPDWDYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGDXGGQGQEPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVVGPMGQPLQVLTLNIEDEYRLHETSKEPDVPLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSEQDCQRGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQAMLLDTDRVQFGPVVALNPATLLPLPEKEAPHDCLEILAETHGTRPDLTDQPIPDADYTWYTDGSSFLQEGQRRAGAAVTTETEVIWARALPAGTSAQRAELIALTQALKMAEGKKLNVYTDSRYAFATAHVHGEIYRRRGLLTSEGREIKNKNEILALLKALFLPKRLSIIHCPGHQKGNSAEARGNRMADQAAREAAMKAVLETSTLLIEDSTPYTPPHFHYTETDLKRLRELGATYNQTKGYWVLQGKPVMPDQSVFELLDSLHRLTHLSPQKMKALLDREESPYYMLNRDRTIQYVTETCTACAQVNASKAKIGAGVRVRGHRPGTHWEVDFTEVKPGLYGYKYLLVFVDTFSGWVEAFPTKRETAKVVSKKLLEDIFPRFGMPQVLGSDNGPAFASQVSQSVADLLGIDWKLHCAYRPQSSGQVERMNRTIKETLTKLTLASGTRDWVLLLPLALYRARNTPGPHGLTPYEILYGAPPPLVNFHDPEMSKLTNSPSLQAHLQALQAVQQEVWKPLAAAYQDQLDQPVIPHPFRVGDAVWVRRHQTKNLEPRWKGPYTVLLTTPTALKVDGISAWIHAAHVKAATTPPAGTAWKVQRSQNPLKIRLTRGAP

SEQ ID NO: 12 - protein, 536 aa, Xenotropic MuLV-related Virus VP62; identical to SEQ ID NO: 9

SEQ ID NO: 13 - protein, 645 aa, Xenotropic MuLV-related Virus VP62; identical to SEQ ID NO: 7

SEQ ID NO: 14 - protein, 1733 aa, Xenotropic MuLV-related Virus VP62; identical to SEQ ID NO: 8
  misc_feature (537)..(537): Xaa can be any naturally occurring amino acid

SEQ ID NO: 15 - protein, 536 aa, Xenotropic MuLV-related Virus VP62; identical to SEQ ID NO: 9

SEQ ID NO: 16 - protein, 409 aa, Friend Spleen Focus-Forming Virus (isolate 502)
MKGPAFSKPLKDKINPWGPLIVLGILIRAGVSVQHDSPHQVFNVTWRVTNLMTGQTANATSLLGTMTDAFPMLHFDLCDLIGDDWDETGLECRTPGGRKRARTFDFYVCPGHTVPTGCGGPREGYCGKWGCETTGQAYWKPSSSWDLISLKRGNTPKDRGPCYDSSVSSGVQGATPGGRCNPLVLKFTDAGKKASWDSPKVWGLRLYRPTGIDPVTRFSLTRQVLNIGPRIPIGPNPVIIGQLPPSRPVQVRLPRPPQPPPTGAASMVPGTAPPSQQPGTGDRLLNLVQGAYQALNLTNPDKTQECWLCLVSGPPYYEGVAVLGTNSNHTSALKEKCCFYADHTGLVRDSMAKLRKRLTQRQKLFESSQGWFEGSFNRSPWFTTLISTIMGLLIILLLLLILLLWTLHS

SEQ ID NO: 17 - protein, 187 aa, Friend Spleen Focus-Forming Virus (isolate 502)
MGQTVTTPLSLTLEHWEDVQRTASNQSVDVKKRRWVTFCSAEWPTFGVGWPQDGTFNLDIILQVKSKVFSPGPHGHPDQVPYIVTWEAIAYEPPPWVKPFVSPKLSPSPTAPILPSGPSTQPPPRSALYPALTPSIKPGPSPIMADLSLTFSQKTLRRTEDRDRPPLTEMATEKRPPPLLRFLPPLP

SEQ ID NO: 18 - protein, 356 aa, Friend Spleen Focus-Forming Virus (strain BB6)
MEGPAFSKPLKDKINPWGPLIVLGILIRAGVSVQRDSPHQVFNVTWRVTNLMTGQTANATSLLGTMTDAFPKLYFDLCDLIGNDWDETRLGCRTPGEGKRARTFDLYVCPGHTVPTGCGGPREGYCGKWGCETTGQAYWKPSSSWDLISLKRGNTPKDRGPCYDSSVSSGVQGATPGGRCNPLVLKFTDAGKKASWDAPKVWGLRLYRSTGTDPVTRFSLTRQVLNIGPRVPIGPNPVISDQLPPSRPAQIMLPRPPQPPPPGTASIVPETAPPSQQPGTRDRLLNLVNKAYQALNLTSPDKTQECWLCLVSRPPYYEGVAVLGTNSNHTTLISTIMGLLIILLLLLILLLWTLHS

SEQ ID NO: 19 - protein, 409 aa, Friend Spleen Focus-Forming Virus (strain Lilly-Steeves)
MEGPASSKPLKDKTNPWGPLIILGILIRAGVSVQLDSPHQVSNVTWRVTNLMTGQTANATSLLGTMTEAFPKLYFDLCDLMGDDWDETGLGCRTPGGRKRARTFDFYVCPGHTVPTGCGGPREGYCGKWGCETTGQAYWKPSSSWDLISLKRGNTPKDQGPCYDSSVSSGVLGATPGGRCNPLVLEFTDAGRKASWDAPKVWGLRLYRSTGTDPVTRFSLTRQVLDIGPRVPIGSNPVTTDQLPLSRPVQTMPPRPLQPPPPGAASIVPETAPPPQQPGAGDRLLNLVDGAYQALNLTNPDKIQECWLCLVSGPPYYEGVVVLGTYFNHTIALKEKCCFYADHTGLVRDSMAKLRKRLTQRQKLFESSRGWFEGSSNRSPWFTTLISAIMGSLIILLLLLILLIWTLYS

SEQ ID NO: 20 - protein, 408 aa, Rauscher Spleen Focus-Forming Virus
MEGPAFSKPLKDKINPWGPLIILGILIRAGVSVQHDSPHQVFNVTWRVTNLMTGQTANATSLLGTMTDAFPKLYFDLCDLIGDDWDETGLGCRTPGGRKRARTFDFYVCPGHTVPTGCGGPREGYCGKWGCETTGQAYWKPSSSWDLISLKRGNTPRNQGPCYDSSAVSSDIKGATPGGRCNPLVLEFTDAGKKASWDGPKVWGLRLYRSTGTDPVTRFSLTRQVLNIGPRVPIGPNPVITDQLPPSRPVQIMLPRPPQPPPPGAASIVPETAPPSQQPGTGDRLLNLVDGAYQALNLTNPDKTQDCWLCLVSGPPYYEGVAVLGTYYNHTSALKEECCFYADHTGLVRDSMAKLRERLTQRQKLFESSQGWFEELFNRSTWFTTLIFTIIGPLIILLLILLFWTLHS

SEQ ID NO: 21 - protein, 116 aa, Rauscher Spleen Focus-Forming Virus
AHLHALYLVHHEVWRPLAAAYQHQLDRPIVPHPFRLGDTVWVRRHQTNNLQPRWKAPYTVLLTTPTALKVDGIAAWIHAAHVKAATTPPAGTASGPTWKVQRSQNPLKIRLTRGAP

SEQ ID NO: 22 - protein, 27 aa, Rauscher Spleen Focus-Forming Virus
YNHTSALKRECCFYADHTGLVRDSMAK

SEQ ID NO: 23 - protein, 408 aa, Rauscher Spleen Focus-Forming Virus; identical to SEQ ID NO: 20

SEQ ID NO: 24 - protein, 116 aa, Rauscher Spleen Focus-Forming Virus; identical to SEQ ID NO: 21

(19) United States
(12) Patent Application Publication
(10) Pub. No.: US 2010/0167268 A1
(43) Pub. Date: Jul. 1, 2010

(54) SEROCONVERSION ASSAYS FOR DETECTING XENOTROPIC MURINE LEUKEMIA VIRUS-RELATED VIRUS

(76) Inventors: Judy A. Mikovits, Reno, NV (US); Francis W. Ruscetti, New Market, MD (US); Sandra K. Ruscetti, New Market, MD (US)

Correspondence Address: SONNENSCHEIN NATH & ROSENTHAL LLP, P.O. BOX 061080, WACKER DRIVE STATION, WILLIS TOWER, CHICAGO, IL, 60606-1080, US

(21) Appl. No.: 12/575,467

(22) Filed: Oct. 7, 2009

Related U.S. Application Data
(60) Provisional application No. 61/225,877, filed on Jul. 15, 2009.

(51) Int. Cl. C12Q 1/70

(52) U.S. Cl. 435/5

(57) ABSTRACT

Methods of detecting, diagnosing, monitoring or managing an XMRV-related disease such as an XMRV-related neuroimmune disease such as chronic fatigue syndrome or an XMRV-related lymphoma such as mantle cell lymphoma in a subject are disclosed. These methods comprise determining presence, absence or quantity of antibodies against XMRV in a sample from a subject.

### CROSS-REFERENCE TO RELATED APPLICATIONS

This application claims priority from U.S. Provisional Application Ser. No. 61/225,877 filed on Jul. 15, 2009, which is incorporated herein by reference in its entirety.

### GOVERNMENT INTERESTS

This work was supported at least in part with funds from the federal government under U.S.P.H.S. Grant HHSN26120080001E and Grant NCI/NIH CA104943 awarded by the National Institutes of Health, and Grant W81XWH-07-1338 awarded by U.S. Department of Defense Prostate Cancer Research Program. The U.S. Government may have certain rights in the invention.

### INCORPORATION-BY-REFERENCE OF MATERIAL SUBMITTED IN COMPUTER READABLE FORM

The Sequence Listing, which is a part of the present disclosure, includes a computer readable form and a written sequence listing comprising nucleotide and/or amino acid sequences of the present disclosure. The sequence listing information recorded in computer readable form is identical to the written sequence listing. The subject matter of the Sequence Listing is incorporated herein by reference in its entirety.

### INTRODUCTION

The present teachings are in the fields of detection of serum antibodies against retroviruses, and diagnosis of human diseases associated with retroviruses.

Multiple Sclerosis (MS), Amyotrophic Lateral Sclerosis (ALS), fibromyalgia, autism and Chronic Fatigue Syndrome (CFS) are examples of neurological diseases believed to involve malfunctions in the immune system. Neurological maladies and upregulation of inflammatory cytokines and chemokines are some of the more commonly reported observations associated with CFS. Retroviral involvement has long been suspected not only for CFS but also for other neurological diseases such as Multiple Sclerosis (MS) and Amyotropic Lateral Sclerosis (ALS) (DeFreitas, E., et al., Proc. Nat'l. Acad. Sci. USA 88: 2922-2926, 1991; Rolland, A., et al., J Neuroimmunol 160: 195-203, 2005; Steele, A. J., et al., Neurology 64, 454-458, 2005). Several retroviruses such as the Murine Leukemia Viruses (MuLVs), primate retroviruses and HTLV-1 are not only associated with cancer but are also associated with neurological diseases (Power., C., Trends in Neurosci. 24: 162-169, 2001). Investigation of the molecular mechanism of retroviral induced neurodegeneration in rodent models revealed vascular and inflammatory changes mediated by cytokines and chemokines and these changes were observed prior to any neurological pathology (Li, X., et al., J. Virol. 83: 4912-4922, 2009; Peterson, K. E., et al., Curr. Topics Microbiol. Immunol. 303: 67-95, 2006).

A lymphoma such as Mantle Cell Lymphoma (MCL) is a follicular lymphoma characterized by proliferation of atypical small lymphoid cells in wide mantles around benign germinal centers. (Weisenburger, D. D., et al., Blood 87: 4483-4494, 1996; Weisenburger, D. D., et al., Cancer 49: 1429-1438, 1982). MCL has been difficult to treat (Zelenetz, A. D., Annals of Oncology 17 (Supplement 4): iv12-iv14, 2006).

The gammaretrovirus Xenotropic Murine Leukemia Virus-Related Virus (XMRV) has recently been implicated in prostate cancers (Dong, B., et al., Proc. Nat'l. Acad. Sci. USA 104, 1865-1660, 2007; PCT patent application PCT/US2006/013167, published as PCT publication number WO2006110589 of Silverman et al.).

McCormick et al. recently explored the candidacy of XMRV in ALS; however, they did not find XMRV in the blood or CSF of the 25 ALS patients where reverse transcriptase (RT) was detected (McCormick, A. L., et al., Neurology 70: 278, 2008).

Villinger et al. (AIDS Research and Human Retroviruses 2009, abstract OP-58, page 60), described seroconversion in Rhesus Macaques in response to XMRV infection using a Western Blot assay; specifically, an antibody response to env and gag proteins was reported in Rhesus Macaques. Based on these results, a double-antigen sandwich assay was developed to detect seroconversion events in both macaque and human. However, these findings have not been extended to detection of seroconversion antibodies against XMRV in humans.

### SUMMARY

The present inventors have developed and disclose herein methods of detecting antibody against the gammaretrovirus Xenotropic Murine Leukemia Virus-Related Virus (XMRV) in a subject. Furthermore, the present inventors have established that a diagnosis of a neurological disease such as a neuroimmune disease, or a lymphoma in a subject can correlate with infection with XMRV. In various aspects, a subject can be a person having, suspected of having, or at risk for developing an XMRV-related disease.

In various aspects of the present teachings, an XMRV-related disease that can be diagnosed using methods of the present teachings can be any disease associated with XMRV infection, such as, for example, an XMRV-related cancer such as a prostate cancer. In some aspects, the XMRV-related disease can be an XMRV-related lymphoma. In further aspects, the XMRV-related lymphoma can be an XMRV-related Mantle Cell Lymphoma (MCL) or an XMRV-related Chronic Lymphocytic Leukemia lymphoma (CLL). In other aspects, an XMRV-related disease can be an XMRV-related neural disease, such as, without limitation, an XMRV-related neuroimmune disease. In various embodiments, an XMRV-related neuroimmune disease can be, without limitation, chronic fatigue syndrome (CFS), Niemann-Pick Type C Disease, fibromyalgia, Multiple Sclerosis (MS), Parkinson's Disease, Amyotrophic Lateral Sclerosis (ALS) or autism. In a some configurations, the Multiple Sclerosis can be Atypical Multiple Sclerosis. In some aspects of the methods, a subject can exhibit signs and/or symptoms of a neuroimmune disease and/or a lymphoma. In some other aspects, an XMRV-related disease can be a neural disease, such as, without limitation, an XMRV-related neural disease that is not generally recognized as a neuroimmune disease, such as, for example, XMRV-related bipolar disorder or a neurodegenerative disease such as XMRV-related Alzheimer's disease or XMRV-related Parkinson's disease.

The present inventors have determined that the presence of antibodies against XMRV in a subject can be diagnostic for, or can aid in the diagnosis of, any XMRV-related disease, including any disease associated with XMRV infection, or for which XMRV infection is implicated or correlated. Accordingly, in various embodiments of the present teachings, detection and/or quantification of antibodies against XMRV in a subject can be used to diagnose an XMRV-related disease, monitor progress of an XMRV-related disease, or determine efficacy of a treatment of an XMRV-related disease in a subject.

The present inventors provide herein methods for detecting antibodies against XMRV in a subject, such as seroconversion antibodies against XMRV. In various embodiments, the methods include detecting presence, absence or quantity of antibodies against XMRV in a in a sample from a subject, such as a sample of a body fluid from a human subject. In various embodiments, a sample of a body fluid from a subject can be, without limitation, a sample of blood, plasma, serum, sputum, or cerebrospinal fluid from the subject. In some embodiments, a blood sample can be a peripheral blood sample. In some embodiments, the methods allow quantification of antibody against XMRV in a sample. Also disclosed are methods for detecting XMRV infection in a subject, and methods of diagnosing an XMRV-related disease in a subject.

In various aspects, methods of the present teachings can comprise detecting the presence or quantity of antibody against at least one gammaretrovirus antigen such as an XMRV antigen in a sample such as a body fluid sample from a subject. In various embodiments, methods disclosed herein include contacting a subject sample with at least one gammaretrovirus antigen in vitro, and detecting binding between the at least one gammaretrovirus antigen and antibody against the at least one gammaretrovirus antigen. Any method of detecting antibody-antigen binding known to skilled artisans can be used to detect antibodies against gammaretrovirus such as XMRV in a sample. These methods include, without limitation, ELISA, Western Blot, radioimmunoassay, immunoprecipitation, fluorescence detection methods, such as flow cytometry/fluorescence-activated cell sorting (FACS) assays. In some aspects, competitive binding assays can be used to detect and/or quantify antibodies against at least one gammaretrovirus antigen such as an XMRV antigen in a sample. In some configurations, a competitive binding assay can include contacting, in the presence of a sample, at least one gammaretrovirus antigen with a probe that binds the at least one antigen, and detecting the extent of binding between the at least one antigen and the probe. A reduction in the amount of binding between the at least one antigen and the probe compared to a control can be indicative of the presence of antibody against XMRV in the sample.

In various configurations, a probe that binds an gammaretrovirus antigen can be, for example, a polyclonal or monoclonal antibody against XMRV, an XMRV antigen, or an antigen of a taxonomically related gammaretrovirus. In some configurations, a probe that binds an gammaretrovirus antigen can be, for example, a polyclonal antibody against a virus that is taxonomically related to XMRV. For example, a probe that binds a gammaretrovirus antigen such as an XMRV antigen can be a polyclonal antibody against a Murine Leukemia Virus such as a Xenotropic Murine Leukemia Virus (Xenotropic MuLV). In some configurations, the polyclonal antibody can be against an NZB Xenotropic MuLV (O'Neill, R. R., et al., J. Virol. 53: 100-106, 1985). In some configurations, a polyclonal antibody against Xenotropic MuLV can be a goat antibody against NZB Xenotropic MuLV. In various embodiments, methods disclosed herein comprise detecting antibody in a sample, wherein the antibody binds at least one gammaretrovirus antigen, which can be at least one of a Gag polypeptide, an Env polypeptide, or a Pol polypeptide. In various embodiments, the at least one gammaretrovirus antigen can be at least one of an Env polypeptide and a Pol polypeptide. In various embodiments, the at least one gammaretrovirus antigen can be an Env polypeptide. In various embodiments, the at least one gammaretrovirus antigen can be an XMRV antigen. In some embodiments, methods of detecting antibody in a sample that binds at least one XMRV polypeptide can comprise detecting or quantifying binding of antibody comprised by a sample to a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with an XMRV polypeptide, such as an XMRV Env polypeptide or an XMRV Gag polypeptide. In various configurations, a polypeptide that can be used to detect antibody in a sample that binds XMRV can be a polypeptide of a retrovirus taxonomically related to XMRV, such as a gammaretrovirus. A polypeptide of a gammaretrovirus of these embodiments can be, without limitation, an Env polypeptide of a retrovirus of the Mammalian virus group. In some configurations of these embodiments, a polypeptide can be an Env polypeptide of a murine leukemia-related retrovirus, or an Env polypeptide of a gammaretrovirus such as a spleen focus-forming virus such as a Friend spleen focus-forming virus (SFFV), or a nonecotropic Murine Leukemia Virus such as a polytropic (Pmv) or a modified polytropic (Mmpv) (DeFreitas, E., et al., Proc. Nat'l. Acad. Sci. USA 88: 2922-2926, 1991). Accordingly, various assays for detecting antibody against XMRV in a sample can comprise contacting a sample with an retrovirus polypeptide or a gammaretrovirus polypeptide such as an Env polypeptide, which can be, for example, an XMRV Env polypeptide or an SFFV Env polypeptide, and detecting binding of antibody in the sample to the polypeptide. In some configurations, the methods can comprise detecting binding of antibody in a sample to a polypeptide comprising a contiguous sequence of at least 4 amino acids, at least 5 amino acids, at least 6 amino acids, at least 7 amino acids, at least 8 amino acids, at least 9 amino acids, or at least 10 amino acids of a polypeptide of XMRV or a taxonomically related retrovirus such as a gammaretrovirus, such as, without limitation, an SFFV. Exemplary sequences of gammaretroviral Env, Gag and Pol proteins are set forth in the sequence listings, and can be summarized as follows:

| SEQ | NCBI | | |
|---|---|---|---|
| ID NO: | Accession: | Virus/Isolate | Protein |
| 1 | ABB83226 | Xenotropic MuLV- | putative envelope |
| | | related virus VP35 | polyprotein |
| 2 | ABB83225 | Xenotropic MuLV- | putative gag-pro- |
| | | related virus VP35 | pol polyprotein |
| 3 | ABB83224 | Xenotropic MuLV- | putative gag |
| | | related virus VP35 | polyprotein |
| 4 | ABB83229 | Xenotropic MuLV- | putative envelope |
| | | related virus VP42 | polyprotein |
| 5 | ABB83228 | Xenotropic MuLV- | putative gag-pro- |
| | | related virus VP42 | pol polyprotein |
| 6 | ABB83227 | Xenotropic MuLV- | putative gag |
| | | related virus VP42 | polyprotein |
| 7 | YP_512363 | Xenotropic MuLV- | putative envelope |
| | | related virus VP62 | polyprotein |
| 8 | YP_512361 | Xenotropic MuLV- | putative gag-pro- |
| | | related virus VP62 | pol polyprotein |
| 9 | YP_512362 | Xenotropic MuLV- | putative gag |
| | | related virus VP62 | polyprotein |
| 10 | ABM47429 | Xenotropic MuLV- | putative envelope |
| | | related virus VP62 | glycoprotein |
| 11 | ABM47428 | Xenotropic MuLV- | putative gag-pro- |
| | | related virus VP62 | pol polyprotein |
| 12 | ABM47427 | Xenotropic MuLV- | putative gag |
| | | related virus VP62 | polyprotein |
| 13 | ABD49688 | Xenotropic MuLV- | putative envelope |
| | | related virus VP62 | polyprotein |
| 14 | ABD49687 | Xenotropic MuLV- | putative gag-pro- |
| | | related virus VP62 | pol polyprotein |
| 15 | ABD49686 | Xenotropic MuLV- | putative gag |
| | | related virus VP62 | polyprotein |
| 16 | P03393 | Friend spleen focus- | putative env |
| | | forming virus | polyprotein |
| | | (isolate 502) | |
| 17 | P03331 | Friend spleen focus- | putative gag/core |
| | | forming virus | polyprotein |
| | | (isolate 502) | |
| 18 | P31793 | Friend spleen focus- | putative env |
| | | forming virus | polyprotein |
| | | (strain BB6) | |
| 19 | P03394 | Friend spleen focus- | putative env |
| | | forming virus | polyprotein |
| | | (strain Lilly-Steeves) | |
| 20 | P03389 | Rauscher spleen focus- | putative env |
| | | forming virus | polyprotein |
| 21 | P03358 | Rauscher spleen focus- | putative pol |
| | | forming virus | polyprotein |
| 22 | AAA46506 | Rauscher spleen focus- | env polyprotein |
| | | forming virus | |
| 23 | AAA46505 | Rauscher spleen focus- | gp54 precursor |
| | | forming virus | peptide (env) |
| 24 | AAA46504 | Rauscher spleen focus- | polymerase (pol) |
| | | forming virus | |

In various configurations, a gammaretrovirus polypeptide that can be used in aspects of the present methods can be an Env polypeptide or a portion thereof comprising a sequence of at least 4 contiguous amino acids of an Env polypeptide, at least 5 contiguous amino acids of an Env polypeptide, at least 6 contiguous amino acids of an Env polypeptide, at least 7 contiguous amino acids of an Env polypeptide, at least 8 contiguous amino acids of an Env polypeptide, at least 9 contiguous amino acids of an Env polypeptide, or at least 10 contiguous amino acids of an Env polypeptide. In various embodiments of the methods, a polypeptide that can be used to detect antibody that binds XMRV can be a fully- or partially-denatured polypeptide such as, for example a fully- or partially-denatured XMRV or a fully- or partially-denatured SFFV Env polypeptide, or can be a fully folded protein such as an XMRV Env protein or an SFFV Env protein. In various configurations, a polypeptide that can be used to detect antibody that binds XMRV in a sample can be comprised by a eukaryotic cell ex vivo, such as a mammalian cell ex vivo or an insect cell ex vivo, or can be encoded by a polynucleotide and expressed in a microorganism, which can be a eukaryotic microorganism such as a yeast, or a prokaryotic microorganism such as an E. coli. In some configurations, a eukaryotic cell that expresses a polypeptide of the present teachings ex vivo can express the polypeptide on the cell surface or in the cytoplasm, or can secrete the polypeptide. In some embodiments, a peptide can be synthesized using chemical synthesis methods known to skilled artisans such as solid phase synthesis methods of Merrifield (see, e.g., Merrifield, R. B., J. Am. Chem. Soc. 85: 2149-2154, 1963; Marshall, G. R. and R. B. Merrifield, Peptides prepared by solid-phase synthesis. In: CRC Handbook of Biochemistry, H. A. Sober, Ed., Chemical Rubber Co., 2nd Ed., Cleveland Ohio, 1970).

In some embodiments of the methods, a polypeptide that can be used to detect antibody against XMRV can be an antigen other than a Gag polypeptide of a gammaretrovirus such as XMRV or SFFV, such as a p30 Gag polypeptide, a p15 Gag polypeptide or a p10 Gag polypeptide. In other embodiments, methods of detecting the presence, absence or quantity of antibody can be other than a double antigen sandwich assay (Qiu, X., et al., J Med Virol. 80: 484-493, 2008).

In additional embodiments, some methods of the present teachings include providing at least one cell ex vivo that comprises an antigen that can be used to detect antibody against XMRV in a sample. In some configurations, a cell ex vivo can be a mammalian cell expressing at least one gammaretrovirus antigen such as an XMRV antigen or an SFFV antigen ex vivo. The antigen can be, for example, an Env antigen of SFFV. In some configurations, a mammalian cell can be a pro-B cell such as a BaF3 cell (ATCC) or a BaF3ER cell (comprising an erythropoietin receptor). In some alternative configurations, a mammalian cell expressing at least one XMRV antigen can be a BaF3ER-SFFVEnV cell expressing Env protein of Friend spleen focus-forming virus (SFFV Env antigen).

In additional embodiments, some methods of the present teachings include providing a solid support comprising an antigen that can be used to detect antibody against XMRV in a sample. A solid support can be, for example, a bead, a particle, or an ELISA plate, and an XMRV antigen can be adsorbed or attached to the support, e.g., through a covalent attachment. In some configurations, a cross-linking agent can be used to attach an XMRV antigen to a solid support.

In various aspects, the methods of the present teachings can comprise providing a biological sample such as a fluid sample from the subject, and forming a mixture comprising at least one XMRV antigen, the sample, and a competitive probe against the at least one XMRV antigen. In various configurations, the mixture can be subjected to conditions sufficient for formation of a complex comprising the at least one XMRV antigen and the competitive probe. The methods can further comprise detecting the quantity of a complex comprising the at least one XMRV antigen and the competitive probe. In various configurations of the methods, if the sample comprises antibody against the XMRV antigen, the quantity of complex will be less than that of a complex formed from a mixture comprising the XMRV antigen, the competitive probe, and a control sample not comprising antibody against the XMRV antigen. Hence, quantification of a complex comprising the at least one XMRV antigen and the competitive probe can be used to determine the presence, absence or quantity of antibody against XMRV in the sample.

In various aspects, the competitive probe can be, for example, an antibody against a retrovirus antigen such as a gammaretrovirus antigen. For example, a competitive probe can be an anti gp 55 Env antibody. In some configurations, the competitive probe can be monoclonal antibody MAb 7C10 against SFFV Env antigen (Wolff, L. et al., J. Virol. 3: 72-81 (1982)). In an alternative embodiment, the competitive probe can be a polyclonal antibody against an XMRV antigen such as an Env polypeptide.

In various aspects, the present methods of detecting, diagnosing, monitoring or managing an XMRV-related disease in a subject can also comprise providing a biological sample such as a fluid sample from the subject; forming a mixture comprising a) at least one gammaretrovirus antigen such as an XMRV antigen and b) the sample, under conditions sufficient for formation of a complex comprising the at least one antigen and antibody against the at least one XMRV antigen if present; and detecting presence, absence or quantity of a complex comprising the at least one XMRV antigen and antibody against the at least one XMRV antigen.

In various configurations of the methods, the detecting presence, absence or quantity of a complex comprising the at least one gammaretrovirus antigen such as an XMRV antigen and antibody against the at least one antigen can comprise contacting the mixture with at least one primary probe directed against antibody from the subject, such as an antibody against human immunoglobulin. In various aspects, the primary probe can be a polyclonal antibody or a monoclonal antibody. In further configurations, the primary probe can be selected from the group consisting of an antibody, an antigen-binding fragment of an antibody, an aptamer, and an avimer. In an alternative configuration, the primary probe can be an antibody or an antigen-binding fragment thereof. In some configurations, an antigen-binding fragment can be an Fab fragment. In further configurations, the antibody can be an anti gp 55 Env antibody. In an alternative configuration, the antibody can be monoclonal antibody MAb 7C10 (Wolff, L. et al., J. Virol. 3: 72-81 (1982)). In alternative embodiments, the antibody can be a polyclonal antibody against a gammaretrovirus antigen such as, without limitation, an XMRV antigen or an SFFV antigen.

In some additional embodiments, detection of a complex can comprise an immune detection assay such as, without limitation, an immunoprecipitation assay, an ELISA, a radioimmunoassay, a Western blot assay or a flow cytometry assay. In some configurations, detection of a complex can comprise a flow cytometry assay.

In some additional aspects, a probe can comprise a label, and the detecting presence, absence or quantity of a complex can comprise quantifying the label. In some configurations, the label can be an enzyme, a radioisotope, a fluorogen, a fluorophore, a chromogen or a chromophore.

When a label is an enzyme, the enzyme can be any enzyme for which a substrate is available. Examples of such enzymes include, without limitation, a chloramphenicol acetyl transferase, a peroxidase such as a horseradish peroxidase, a phosphatase such as an alkaline phosphatase, a galactosidase such as a β-galactosidease, a β-glucoronidase and a luciferase, such as a firefly luciferase or a renilla luciferase. In some configurations, an alkaline phosphatase can be a secreted alkaline phosphatase. In some configurations, a substrate can be a chromogen or a fluorogen, or can yield a chemiluminescent product. If the substrate is a chemiluminescent substrate, qualititative and/or quantitative detection of the enzyme can comprise measuring light produced as a product of a reaction between the substrate and the enzyme. For example, if the enzyme is an alkaline phosphatase, the substrate can be a chemiluminescent substrate such as CDP-Star® (Sigma-Aldrich Chemical Co., St. Louis, Mo.). In another example, if the enzyme is a luciferase, the substrate can be a luciferin. If the substrate is a chromogenic substrate, qualititative and/or quantitative detection of the enzyme can comprise visual assessment, and/or measuring optical absorbance of the reaction product, such as, without limitation, measuring absorbance at 400 nm when the enzyme is an alkaline phosphatase and the substrate is dinitrophenyl phosphate. If the substrate is a fluorogenic substrate, qualititative and/or quantitative detection of the enzyme can comprise visual assessment, and/or measuring fluorescent light intensity using a fluorometer.

In some configurations, when the label is a chromophore, the label can be any chromophore known to skilled artisans, such as, without limitation, a dichlorotriazine dye such as 1 Amino 4[3(4,6 dichlorotriazin 2 ylamino) 4 sulfophenylamino]anthraquinone 2 sulfonic acid (Procion Blue MX R® (Fluka A G, Switzerland)). Such labels can be detected by methods known to skilled artisans, such as measurement of optical absorbance using a spectrophotometer.

In some configurations, when the label is a fluorophore, the label can be any fluorophore known to skilled artisans, such as, without limitation, a fluorescein, a rhodamine, an Alexa Fluor® (Invitrogen Corporation, Carlsbad, Calif.) a coumarin, an indocyanine or a quantum dot (Colton, H. M., et al., Toxicological Sciences 80: 183 192, 2004). In addition, in some configurations a fluorophore can be a fluorescent protein, such as a phycoerythrin or a green fluorescent protein. Such fluorescent labels can be detected by methods known to skilled artisans, such as fluorescence microscopy or measurement of fluorescence using a fluorometer or a flow cytometry apparatus.

In some configurations, when the label is a radioisotope, the radioisotope can be any radioisotope known to skilled artisans, such as, without limitation, a ³²P, a ³³P, ³⁵S, a ¹⁴C, an ¹²⁵I, an ¹³¹I or a ³H. In some configurations, the enzyme can be a peroxidase, a phosphatase, a galactosidase or a luciferase.

In yet other configurations, the label can be a probe-binding target such as a biotin, a digoxygenin, or a peptide comprising an epitope. In some configurations, when the label is a probe binding target, the probe binding target can be any molecular target for a probe, such as, without limitation, a ligand to which a probe binds, such as, without limitation, an antigen which an antibody binds. In various configurations of these methods, a probe binding target can be, without limitation, a biotin, a digoxygenin, or a peptide, and a probe for the probe binding target can be, without limitation, an avidin, a streptavidin, an anti biotin antibody, an anti digoxygenin antibody, or a peptide antibody directed against a peptide. Accordingly, in various configurations of these methods, a label and a probe can be, without limitation, a) a biotin and an avidin, b) a biotin and a streptavidin, c) a biotin and an anti biotin antibody, d) a digoxygenin and an anti digoxygenin antibody, or e) a peptide and an antibody directed against the peptide.

In additional embodiments, methods of the present teachings can further comprise selecting or modifying a treatment on the basis of the detection of antibody against XMRV in a sample from a subject. In various aspects, if antibody against XMRV is detected in the sample, the treatment can comprise administrating to the subject a therapeutically effective amount of an anti-viral compound. In some configurations, the antiviral compound can be, without limitation, a compound such as acyclovir, penciclovir (famciclovir), gancyclovir (ganciclovir), deoxyguanosine, foscarnet, idoxuridine, trifluorothymidine, vidarabine, sorivudine, zidovudine, didanosine, zalcitabine, lamivudine, stavudine, abacavir, multinucleoside resistance A, multinucleoside resistance B, nevirapine, delavirdine, efavirenz, adefovir dipivoxil, indinavir, ritonavir, saquinavir, nelfinavir, amprenavir, deoxycytosine triphosphate, lamivudine triphosphate, emticitabine triphosphate, adefovir diphosphate, penciclovir triphosphate, lobucavir triphosphate, amantadine, rimantadine, zanamivir or oseltamivir.

### BRIEF DESCRIPTION OF THE DRAWINGS

FIG. 1. illustrates presence of antibodies to SFFV-env in CFS patients' plasma.

FIG. 2 illustrates that antibodies in CFS plasma recognize cell surface SFFV Env expressed in cell line BaF-3.

FIG. 3 illustrates antibody reactivity in CFS plasma to SFFV-Env expressed in BAF3ER cells.

### DETAILED DESCRIPTION

In the present teachings, the inventors set forth methods which can be used to detect antibodies against XMRV. The present disclosure demonstrates identification of antibodies against XMRV in humans and provides methods for identification of this virus. As used herein, an “XMRV antibody” includes an antibody that binds to XMRV or at least one molecular component thereof, such as, without limitation, a gag protein, an env protein, or a pol protein. An XMRV antibody can also be cross-reactive against a retrovirus in addition to XMRV or at least one antigenic component thereof, such as a gammaretrovirus. A gammaretrovirus to which a human XMRV antibody can be cross-reactive can be, without limitation, a spleen focus-forming virus, including a Friend spleen focus-forming virus, or a retrovirus within the Mammalian retrovirus group. This retrovirus group includes various murine leukemia-related retroviruses in addition to an XMRV, such as, without limitation, an Epicrionops marmoratus retrovirus, an Ichthyophis kohtaoensis retrovirus, an Osteolaemus tetraspis retrovirus, a Sericulus bakeri retrovirus, a Terdus iliacus retrovirus, a Tomistoma schlegelii retrovirus, and a Viper berus retrovirus.

Methods and compositions described herein utilize laboratory techniques well known to skilled artisans. Such techniques can be found in laboratory manuals such as Sambrook, J., et al., Molecular Cloning: A Laboratory Manual, 3rd ed. Cold Spring Harbor Laboratory Press, Cold Spring Harbor, N.Y., 2001; Spector, D. L. et al., Cells: A Laboratory Manual, Cold Spring Harbor Laboratory Press, Cold Spring Harbor, N.Y., 1998; Harlow, E., Using Antibodies: A Laboratory Manual, Cold Spring Harbor Laboratory Press, Cold Spring Harbor, N.Y., 1999. Methods of administration of pharmaceuticals and dosage regimes, can be determined according to standard principles of pharmacology well known skilled artisans, using methods provided by standard reference texts such as Remington: the Science and Practice of Pharmacy (Alfonso R. Gennaro ed. 19th ed. 1995); Hardman, J. G., et al., Goodman & Gilman's The Pharmacological Basis of Therapeutics, Ninth Edition, McGraw-Hill, 1996; and Rowe, R. C., et al., Handbook of Pharmaceutical Excipients, Fourth Edition, Pharmaceutical Press, 2003. These publications are incorporated herein by reference, each in its entirety.

As used herein, the singular forms “a”, “an” and “the” are intended to include the plural forms as well, unless the context indicates otherwise. The following examples are illustrative and are not intended to be limiting to the scope of any claim.

### EXAMPLES

Various experiments presented in the Examples utilize the following materials and methods for flow cytometry for detection of antiviral antibodies in CFS plasma: Murine cell lines BaF3ER and BAF3ER-SFFV Env (Nishigaki, K., et al., J. Virol. 75: 7893-7903, 2001) comprising gp55 env plasmid to express SFFV gp 55 env were grown in 2 units/ml of Epo in RPMI 1640 in 7% FCS. 500,000 cells per sample in log phase were used as targets for direct staining Cell lines were first washed in wash buffer (2% FBS, 0.02% Na Azide, PBS) and resuspended in 200 μl of BSA staining buffer (BD PharMingen, San Jose, Calif.). Patient plasma was thawed rapidly and used at 20 μl or 2 μl per tube (1:10 and 1:100 respectively). Samples were incubated at 4° C. or on ice for 30 minutes. Cells were then washed with 0.5 mL of the wash buffer. Tubes were centrifuged at 800 rpm for 5 minutes, the supernatant was removed and samples were blotted on a towel. Next, 100 μL of the following working solution was added: 5 μL human A/B sera, 1 μL at biotin-labeled anti-human IgG (for human plasma or biotin-labeled anti rat IgG (for 7C10 monoclonal antibody against SFFV Env, Wolff, L. et al., J. Virol. 3: 72-81 (1982)) (Ebioscience, San Diego, Calif.), 1 μL at of strep/avidin phycoerythrin (PE), 94 μL cold staining buffer. Samples were then incubated at 4° C. for 20 minutes, washed with 0.5 mL of the wash buffer, and spun at 800 rpm for 5 minutes before being analyzed by flow cytometry. For the competition experiments, 100 μL of cold staining buffer and 10 μL of human plasma were added to each tube prior to addition of either anti-SFFV Env mAb (7c10) or Y3 myeloma supernatant (control). Samples were incubated at 4° C. or on ice for 20 minutes, washed with 0.5 mL of wash buffer and spun at 800 rpm for 5 minutes before being analyzed by flow cytometry.

Patient samples: Banked samples were selected for this study from patients fulfilling the 1994 CDC Fukuda Criteria for Chronic Fatigue Syndrome (Fukuda, K., et al., Ann. Intern. Med. 121: 953-959, 1994) and the 2003 Canadian Consensus Criteria for Chronic Fatigue Syndrome/myalgic encephalomyelitis (CFS/ME) and presenting with severe disability. Samples were selected from several regions of the United States where outbreaks of CFS had been documented (DeFreitas, E., et al., Proc. Nat'l. Acad. Sci. USA 88: 2922-2926, 1991). These are patients that have been seen in private medical practices, and their diagnosis of CFS is based upon prolonged disabling fatigue and the presence of cognitive deficits and reproducible immunological abnormalities. These included but were not limited to perturbations of the 2-5A synthetase/RNase L antiviral pathway, low natural killer cell cytotoxicity (as measured by standard diagnostic assays), and elevated cytokines particularly interleukin-6 and interleukin-8. In addition to these immunological abnormalities, the patients characteristically demonstrated impaired exercise performance with extremely low VO2 max measured on stress testing. The patients had been seen over a prolonged period of time and multiple longitudinal observations of the clinical and laboratory abnormalities had been documented.

### Example 1

This example illustrates direct detection of antibody against XMRV in plasma from CFS patients.

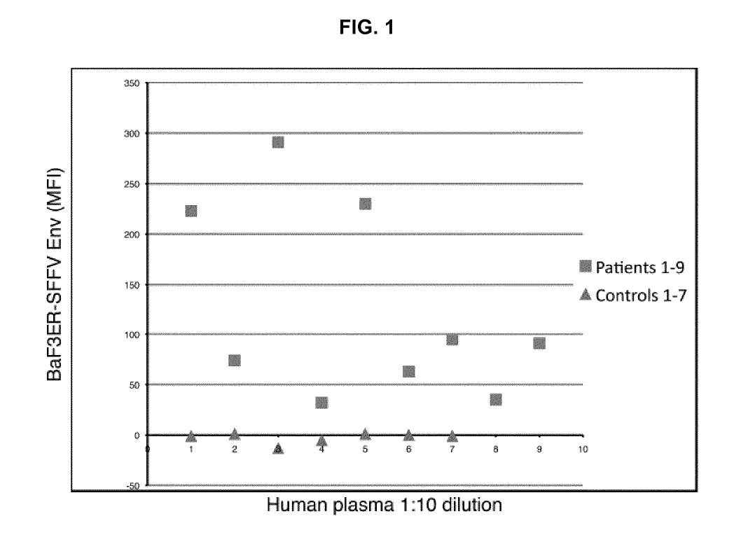

Our prior demonstration that infectious virus is present in both T and B lymphocytes from CFS patients is consistent with the tropism of other well-documented targets of human retroviral infection (B. J. Poiesz et al., Proc Natl Acad Sci USA 77, 7415 1980; J. C. Chemann et al., Antibiot Chemother 32, 48, 1983). We investigated whether XMRV stimulated an immune response in these patients, and developed an assay for detecting antibodies to XMRV ENV. This assay uses a murine pro B cell line, BAF-3 (control) and BAF-3 stably expressing SFFVgp55 ENV. In these experiments, Plasma from CFS patients or normal healthy controls was diluted 1:10, reacted with BaF3-ER or BaF3ERSFFV Env cells and analyzed by intracellular flow cytometry (IFC). FIG. 1 shows the difference in mean fluorescence intensity (MFI) between CFS and control plasma direct binding to BaF3ER-SFFV Env cells versus BaF3ER (control) cells. Direct binding was observed in 9/18 CFS patients positive for XMRV plasma and 0/7 normal plasma.

### Example 2

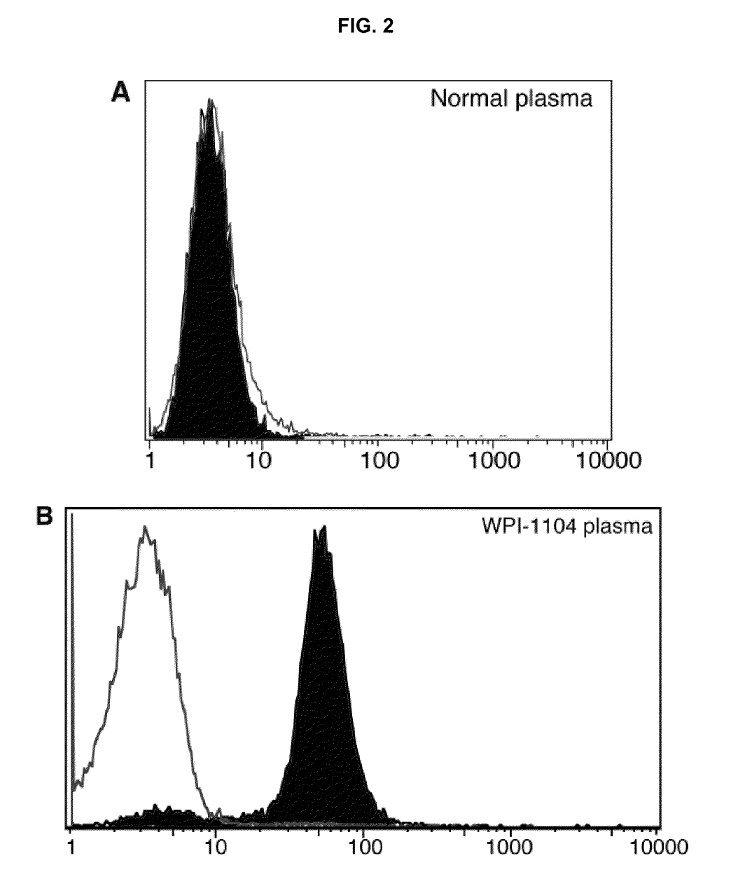

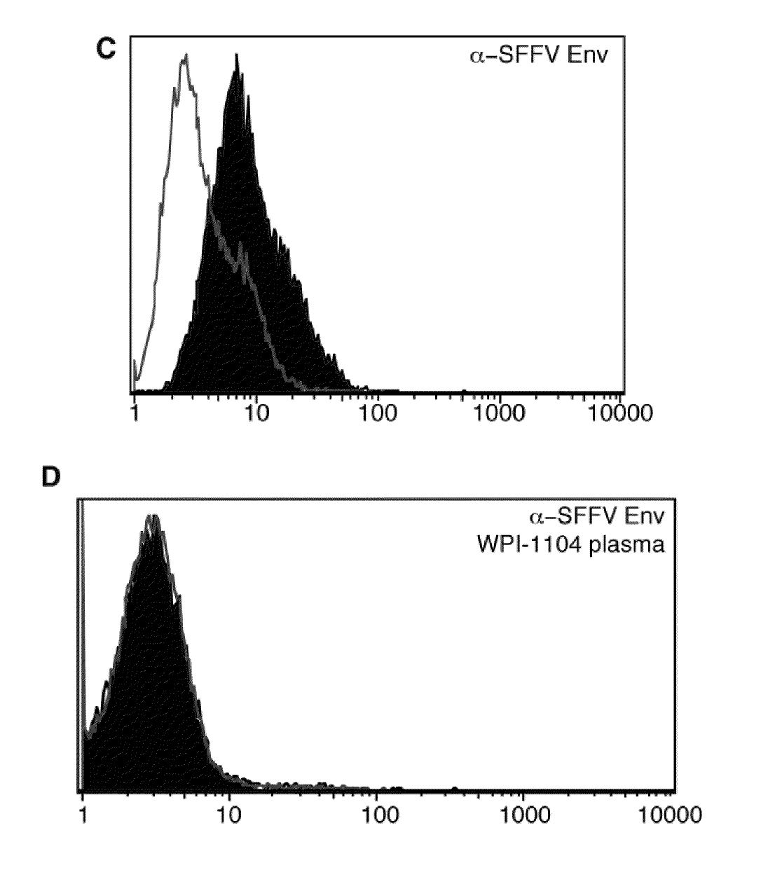

This example illustrates detection of antibody against XMRV in plasma from CFS patients. In these experiments, as illustrated in FIG. 2, samples of human plasma were assayed by flow cytometry for presence of antibody against XMRV using direct and competitive assays.

Direct assay of binding of normal plasma with BaF-3-SFFV ENV was negative (FIG. 2A). Furthermore, as shown in FIG. 2B, direct assay of binding of normal plasma with BaF-3 ER FC was also negative. However, a plasma sample from a patient who was previously diagnosed clinically with CFS (designated patient 1104 by the Whittemore Peterson Institute), was found to comprise antibody against XMRV: as shown in FIG. 2B, black area, a 1:10 dilution of plasma was positive in this assay. FIG. 2C illustrates direct binding of 2 μL 7C10 monoclonal anti SFFV Env antibody to BAF3-SFFV gp55 cell line (black area) vs. binding to BaF-3 ER control (light area). FIG. 2D illustrates competition for binding of 7C10 to BAF3-SFFV gp55 cell line by 1:10 dilution of plasma from patient 1104. These data show specificity of antibody in CFS patient 1104 plasma, and demonstrate the ability of human sera to block 7C10 binding (black area totally overlaps light negative area).

### Example 3

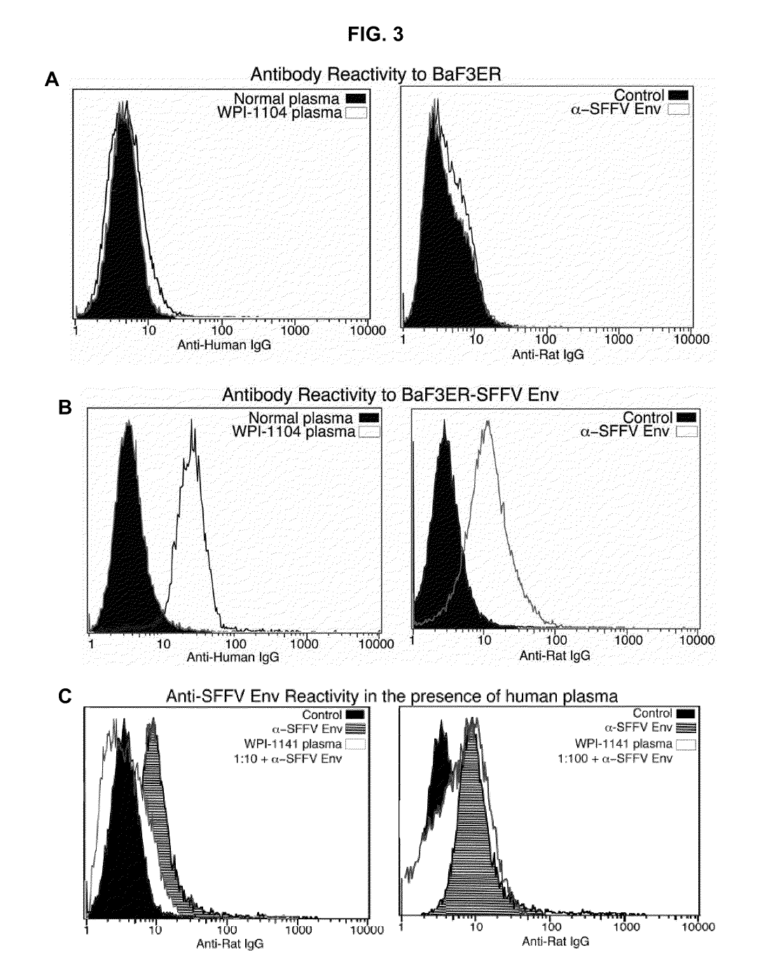

This example illustrates detection of antibodies against XMRV in sera of patients, using direct and competitive assays (FIG. 3).

We investigated whether XMRV stimulates an immune response in CFS patients. For this purpose, we developed a flow cytometry assay that allowed us to detect antibodies to XMRV Env by exploiting its close homology to SFFV Env (Wolff, L., et al., Proc. Nat'l. Acad. Sci. USA 80: 4718-4722, 1983).

FIG. 3A illustrates no direct binding on BAF3ER control cells. Left panel: binding of human plasma at 1:10 dilution as detected by anti human IgG; right panel: no binding of anti-SFFV env monoclonal (7C10) at 1:10 dilution as detected using an anti rat IgG. Y3 rat hybridoma supernatant served as control. FIG. 3B illustrates direct binding on BAF3ER-SFFV Env cells. Left panel illustrates direct binding of human CFS from patient 1104 but not normal plasma at 1:10 dilution as detected by anti human IgG; right panel illustrates direct binding of anti-SFFV Env monoclonal at 1:10 as detected by anti rat IgG. In these experiments, plasma from 9 out of 18 CFS patients infected with XMRV reacted with a mouse B cell line expressing recombinant SFFV Env (BaF3ER-SFFV-Env) (FIG. 3B) but not to SFFV Env negative control cells (BaF3ER) (FIG. 3A), analogous to the binding of the SFFV Env mAb to these cells (FIG. 3A-B, FIG. 1). In contrast, plasma from seven healthy donors did not react (FIG. 3A, FIG. 1).

Further experiments indicate that plasma from a CFS patient can block binding of a rat anti-SFFV Env mAb to BaF3ER-SFFV Env cells. In these experiments, all nine positive plasma samples from CFS patients but none of the plasma samples from healthy donors blocked the binding of the SFFV Env mAb to SFFV Env on the cell surface. As shown in FIG. 3C, CFS plasma competes with anti-SFFV Env for binding to BAF3ER-SFFV Env cells. Left panel: CFS plasma from patient 1141, diluted 1:10 (white area) eliminates most of the anti-SFFV Env binding (striped area) and overlaps with the negative control (black area). Right panel: CFS plasma diluted 1:100 (white area) eliminates less of the anti-SFFV Env binding (striped area) and overlaps much more with the positive than the negative control (black area). We found that at dilutions of 1:10 and 1:100, plasma from several of the CFS patients' but not normal plasma significantly blocked anti-SFFV antibody binding. These experiments show that plasma from a CFS patient can compete with an anti-SFFV Env mAb for binding to cells comprising SFFV Env.

These data indicate that CFS patients mount a specific immune response to XMRV. Furthermore, our results demonstrate that we can reliably detect antibody against XMRV in infected individuals using the disclosed methods, including individuals having an XMRV-related disease such as CFS.

All publications, patent applications, patents, and other references mentioned herein are incorporated by reference, each in its entirety.

The present teachings include the following aspects:

1. A method of detecting presence, absence or quantity of antibody against XMRV in a subject, the method comprising:

providing a biological sample comprising antibody from the subject;

forming a mixture comprising a) at least one gammaretrovirus antigen, b) the sample, and c) a competitive probe against the at least one Gammaretrovirus antigen, under conditions sufficient for formation of a complex comprising the at least one Gammaretrovirus antigen and the competitive probe; and

detecting quantity of a complex comprising the at least one Gammaretrovirus antigen and the competitive probe, whereby if the sample comprises antibody against the Gammaretrovirus antigen, the quantity of complex is less than that of a complex formed from a mixture comprising a) the at least one Gammaretrovirus antigen, b) a control sample not comprising antibody against the at least one Gammaretrovirus antigen, and c) the competitive probe against the at least one Gammaretrovirus antigen.

2. A method in accordance with Aspect 1, wherein the at least one Gammaretrovirus antigen is other than an XMRV gag polypeptide.

3. A method in accordance with Aspect 2, wherein the XMRV gag polypeptide is selected from the group consisting of a p10 polypeptide, a p15 polypeptide and a p30 polypeptide.

4. A method in accordance with Aspect 3, wherein the XMRV gag polypeptide is a p30 polypeptide.

5. A method in accordance with Aspect 1, wherein the method is other than a double antigen sandwich assay.

6. A method in accordance with any one of Aspects 1-5, wherein the subject is a human.

7. A method in accordance with any one of Aspects 1-5, wherein the subject is a person having, suspected of having, or at risk for developing an XMRV-related disease.

8. A method in accordance with Aspect 7, wherein the XMRV-related disease is an XMRV-related prostate cancer.

9. A method in accordance with Aspect 7, wherein the XMRV-related disease is an XMRV-related lymphoma.

10. A method in accordance with Aspect 9, wherein the XMRV-related lymphoma is selected from the group consisting of an XMRV-related Mantle Cell Lymphoma (MCL) and an XMRV-related Chronic Lymphocytic Leukemia lymphoma (CLL).

11. A method in accordance with Aspect 6, wherein the XMRV-related disease is an XMRV-related neuroimmune disease.

12. A method in accordance with Aspect 11, wherein the XMRV-related neuroimmune disease is selected from the group consisting of chronic fatigue syndrome (CFS), Niemann-Pick Type C Disease, fibromyalgia, Multiple Sclerosis (MS), Parkinson's Disease, Amyotrophic Lateral Sclerosis (ALS) and autism.

13. A method in accordance with Aspect 11, wherein the XMRV-related neuroimmune disease is chronic fatigue syndrome (CFS).

14. A method in accordance with Aspect 12, wherein the Multiple Sclerosis is Atypical Multiple Sclerosis.

15. A method in accordance with Aspect 6, wherein the subject exhibits signs and/or symptoms of a neuroimmune disease and/or a lymphoma.

16. A method in accordance with Aspect 1, wherein the sample is selected from the group consisting of a blood sample, a serum sample, a plasma sample, a sputum sample and a cerebrospinal fluid sample.

17. A method in accordance with Aspect 1, wherein the sample is selected from the group consisting of a blood sample, a serum sample, a plasma sample and a cerebrospinal fluid sample.

18. A method in accordance with Aspect 1, wherein the sample is selected from the group consisting of a blood sample, a serum sample and a plasma sample.

19. A method in accordance with any one of Aspect 16, 17 or 18, wherein the sample is a plasma sample.

20. A method in accordance with any one of Aspect 16, 17 or 18, wherein the blood sample is a peripheral blood sample.

21. A method in accordance with Aspect 1, wherein the at least one Gammaretrovirus antigen comprises a contiguous sequence of at least 4 amino acids of an XMRV polypeptide.

22. A method in accordance with Aspect 1, wherein the at least one Gammaretrovirus antigen is comprised by at least one cell ex vivo.

23. A method in accordance with Aspect 22, wherein the at least one cell ex vivo is a mammalian cell expressing at least one Gammaretrovirus antigen ex vivo.

24. A method in accordance with Aspect 22, wherein the at least one Gammaretrovirus antigen is a polypeptide having at least 95% sequence identity with an SFFV Env protein.

25. A method in accordance with Aspect 22, wherein the at least one Gammaretrovirus antigen is a polypeptide having at least 96% sequence identity with an SFFV Env protein.

26. A method in accordance with Aspect 22, wherein the at least one Gammaretrovirus antigen is a polypeptide having at least 97% sequence identity with an SFFV Env protein.

27. A method in accordance with Aspect 22, wherein the at least one Gammaretrovirus antigen is a polypeptide having at least 98% sequence identity with an SFFV Env protein.

28. A method in accordance with Aspect 22, wherein the at least one Gammaretrovirus antigen is a polypeptide having at least 99% sequence identity with an SFFV Env protein.

29. A method in accordance with Aspect 22, wherein the at least one Gammaretrovirus antigen is a polypeptide having 100% sequence identity with an SFFV Env protein.

30. A method in accordance with Aspect 23, wherein the mammalian cell is a BaF3ER cell.

31. A method in accordance with Aspect 23, wherein the mammalian cell expressing at least one Gammaretrovirus antigen is a BaF3ER-SFFVEnV cell expressing SFFV Env protein.

32. A method in accordance with Aspect 1, wherein the at least one Gammaretrovirus antigen is an SFFV antigen.

33. A method in accordance with Aspect 32, wherein the SFFV antigen is an SFFV Env antigen.

34. A method in accordance with any one of Aspects 1-33, wherein the detecting presence, absence or quantity of a complex comprising the at least one Gammaretrovirus antigen and antibody against the at least one Gammaretrovirus antigen comprises contacting the mixture with at least one primary probe directed against antibody from the subject.

35. A method in accordance with claim 34, wherein the at least one primary probe is selected from the group consisting of an antibody, an antigen-binding fragment of an antibody, an aptamer, and an avimer.

36. A method in accordance with Aspect 34, wherein the at least one primary probe is an antibody or an antigen-binding fragment thereof.

37. A method in accordance with Aspect 36, wherein the antibody is a polyclonal antibody or a monoclonal antibody.

38. A method in accordance with Aspect 36, wherein the antigen-binding fragment is an Fab fragment.

39. A method in accordance with Aspect 1, wherein the competitive probe is an antibody against a gammaretrovirus antigen.

40. A method in accordance with Aspect 39, wherein the antibody against a gammaretrovirus antigen is a polyclonal antibody.

41. A method in accordance with Aspect 39, wherein the antibody against a gammaretrovirus antigen is a monoclonal antibody.

42. A method in accordance with any one of Aspects 34-41, wherein the antibody against a gammaretrovirus antigen is an antibody against a murine leukemia-related retrovirus.

43. A method in accordance with any one of Aspects 34-41, wherein the antibody against a gammaretrovirus antigen is an antibody against a Xenotropic murine leukemia virus.

44. A method in accordance with any one of Aspects 34-41, wherein the antibody against a gammaretrovirus antigen is an antibody against a nonecotropic murine leukemia virus.

45. A method in accordance with any one of Aspects 34-41, wherein the antibody against a gammaretrovirus antigen is an antibody against a polytropic murine leukemia virus (Mmpv).

46. A method in accordance with any one of Aspects 34-41, wherein the antibody against a gammaretrovirus antigen is an antibody against a modified polytropic murine leukemia virus.

47. A method in accordance with any one of Aspects 34-41, wherein the antibody against a gammaretrovirus antigen is an antibody against an XMRV.

48. A method in accordance with any one of Aspects 34-41, wherein the antibody against a gammaretrovirus antigen is an antibody against an SFFV.

49. A method in accordance with any one of Aspects 34-48, wherein the antibody against a gammaretrovirus antigen is selected from the group consisting of an antibody against a gammaretrovirus gag protein, an antibody against a gammaretrovirus env protein and an antibody against a gammaretrovirus pol protein.

50. A method in accordance with any one of Aspects 34-48, wherein the an antibody against a gammaretrovirus antigen is selected from the group consisting of an antibody against a gammaretrovirus env protein and an antibody against a gammaretrovirus pol protein.

51. A method in accordance with any one of Aspects 34-48, wherein the an antibody against a gammaretrovirus antigen is an antibody against a gammaretrovirus env protein.

52. A method in accordance with any one of Aspects 34-51, wherein the antibody against a gammaretrovirus antigen is an anti gp 55 Env antibody.

53. A method in accordance with Aspect 51, wherein the antibody against a gammaretrovirus antigen is monoclonal antibody MAb 7C10.

54. A method in accordance with Aspect 36, wherein the antibody is a polyclonal antibody against at least one Gammaretrovirus antigen.

55. A method in accordance with Aspect 36, wherein the detecting comprises an assay selected from the group consisting of an immunoprecipitation assay, an ELISA, a radioimmunoassay, a Western blot assay and a flow cytometry assay.

56. A method in accordance with Aspect 36, wherein the detecting comprises a flow cytometry assay.

57. A method in accordance with Aspect 34, wherein the at least one primary probe comprises a label, and the detecting presence, absence or quantity of the complex comprises quantifying the label.

58. A method in accordance with Aspect 57, wherein the label is selected from the group consisting of an enzyme, a radioisotope, a fluorogen, a fluorophore, a chromogen and a chromophore.

59. A method in accordance with Aspect 58, wherein the enzyme is selected from the group consisting of a peroxidase, a phosphatase, a galactosidase and a luciferase.

60. A method in accordance with Aspect 58, wherein the radioisotope is selected from the group consisting of a ³²P, a ³³P, ³⁵S, a ¹⁴C, an ¹²⁵I, an ¹³¹I and a ³H.

61. A method in accordance with Aspect 58, wherein the fluorophore is selected from the group consisting of a fluorescein, a rhodamine, an Alexa Fluor®, a coumarin, an indocyanine or a quantum dot a phycoerythrin, and a green fluorescent protein.

62. A method in accordance with Aspect 57, wherein the label is selected from the group consisting of a biotin, a digoxygenin, and a peptide comprising an epitope.

63. A method in accordance with Aspect 1, wherein the detecting presence, absence or quantity of a complex comprises:

contacting the complex with at least one secondary probe that binds the at least one primary probe; and

quantifying the at least one secondary probe bound to the complex, whereby if the sample comprises antibody against the Gammaretrovirus antigen, the quantity of the at least one secondary probe bound to the complex is less than that of a complex formed from a mixture comprising a) the at least one Gammaretrovirus antigen, b) a control sample not comprising antibody against the at least one Gammaretrovirus antigen, and c) the competitive probe against the at least one Gammaretrovirus antigen.

64. A method in accordance with Aspect 63, wherein the quantifying the at least one secondary probe comprises an assay selected from the group consisting of an immunoprecipitation assay, an ELISA, a radioimmunoassay, a Western blot assay and a flow cytometry assay.

65. A method in accordance with Aspect 63, wherein the quantifying the at least one secondary probe comprises a flow cytometry assay.

66. A method in accordance with Aspect 1, further comprising selecting or modifying a treatment on the basis of the detection of antibody against XMRV in the sample.

67. A method in accordance with Aspect 66, wherein if antibody against XMRV is detected in the sample, the treatment comprises administrating to the subject a therapeutically effective amount of an anti-viral compound.

68. A method in accordance with Aspect 67, wherein the anti-viral compound is selected from the group consisting of acyclovir, penciclovir (famciclovir), gancyclovir (ganciclovir), deoxyguanosine, foscarnet, idoxuridine, trifluorothymidine, vidarabine, sorivudine, zidovudine, didanosine, zalcitabine, lamivudine, stavudine, abacavir, multinucleoside resistance A, multinucleoside resistance B, nevirapine, delavirdine, efavirenz, adefovir dipivoxil, indinavir, ritonavir, saquinavir, nelfinavir, amprenavir, deoxycytosine triphosphate, lamivudine triphosphate, emticitabine triphosphate, adefovir diphosphate, penciclovir triphosphate, lobucavir triphosphate, amantadine, rimantadine, zanamivir and oseltamivir.

69. A method of detecting, diagnosing, monitoring or managing an XMRV-related disease in a subject, the method comprising:

providing a biological sample comprising antibody from the subject;

forming a mixture comprising a) at least one Gammaretrovirus antigen and b) the sample, under conditions sufficient for formation of a complex comprising the at least one Gammaretrovirus antigen and antibody against the at least one Gammaretrovirus antigen if present in the antibody from the subject; and

detecting presence, absence or quantity of a complex comprising the at least one Gammaretrovirus antigen and antibody against the at least one Gammaretrovirus antigen.

70. A method in accordance with Aspect 69, wherein the at least one Gammaretrovirus antigen is other than a gammaretrovirus gagp30.

71. A method in accordance with Aspect 69, wherein the method is other than a double antigen sandwich assay.

72. A method in accordance with Aspect 69, wherein the subject is a person having, suspected of having, or at risk for developing an XMRV-related disease.

73. A method in accordance with Aspect 72, wherein the XMRV-related disease is an XMRV-related prostate cancer.

74. A method in accordance with Aspect 72, wherein the XMRV-related disease is an XMRV-related lymphoma.

75. A method in accordance with Aspect 74, wherein the XMRV-related lymphoma is selected from the group consisting of a XMRV-related Mantle Cell Lymphoma (MCL) and an XMRV-related Chronic Lymphocytic Leukemia lymphoma (CLL).

76. A method in accordance with Aspect 72, wherein the XMRV-related disease is an XMRV-related neuroimmune disease.

77. A method in accordance with Aspect 76, wherein the XMRV-related neuroimmune disease is selected from the group consisting of chronic fatigue syndrome (CFS), Niemann-Pick Type C Disease, fibromyalgia, Multiple Sclerosis (MS), Parkinson's Disease, Amyotrophic Lateral Sclerosis (ALS) and autism.

78. A method in accordance with Aspect 77, wherein the Multiple Sclerosis is Atypical Multiple Sclerosis.

79. A method in accordance with Aspect 72, wherein the subject exhibits signs and/or symptoms of a neuroimmune disease and/or a lymphoma.

80. A method in accordance with Aspect 69, wherein the sample is selected from the group consisting of a blood sample, a serum sample, a plasma sample, and a cerebrospinal fluid sample.

81. A method in accordance with Aspect 61, wherein the blood sample is a peripheral blood sample.

82. A method in accordance with Aspect 69, wherein the at least one Gammaretrovirus antigen comprises a contiguous sequence of at least 4 amino acids of an XMRV polypeptide.

83. A method in accordance with Aspect 69, wherein the at least one Gammaretrovirus antigen is comprised by at least one cell ex vivo.

84. A method in accordance with Aspect 83, wherein the at least one cell ex vivo is a mammalian cell expressing at least one Gammaretrovirus antigen ex vivo.

85. A method in accordance with Aspect 84, wherein the mammalian cell expressing the at least one gammaretrovirus antigen is a BaF3ER cell.

86. A method in accordance with Aspect 84, wherein the mammalian cell expressing at least one Gammaretrovirus antigen is a BaF3ER-SFFVEnV cell expressing SFFV Env protein.

87. A method in accordance with Aspect 69, wherein the at least one Gammaretrovirus antigen is an SFFV antigen.

88. A method in accordance with Aspect 87, wherein the SFFV antigen is an SFFV Env antigen.

89. A method in accordance with Aspect 69, wherein the detecting presence, absence or quantity of a complex comprising the at least one Gammaretrovirus antigen and antibody against the at least one Gammaretrovirus antigen comprises contacting the mixture with at least one primary probe directed against antibody from the subject.

90. A method in accordance with claim 89, wherein the at least one primary probe is selected from the group consisting of an antibody, an antigen-binding fragment of an antibody, an aptamer, and an avimer.

91. A method in accordance with Aspect 89, wherein the at least one primary probe is an antibody or an antigen-binding fragment thereof c92. A method in accordance with Aspect 91, wherein the antibody is a polyclonal antibody or a monoclonal antibody.

93. A method in accordance with Aspect 91, wherein the antigen-binding fragment is an Fab fragment.

94. A method in accordance with Aspect 91, wherein the antibody is an anti gp 55 Env antibody.

95. A method in accordance with Aspect 91, wherein the antibody is a monoclonal antibody MAb 7C10.

96. A method in accordance with Aspect 91, wherein the antibody is a polyclonal antibody against at least one Gammaretrovirus antigen.

97. A method in accordance with Aspect 91, wherein the detecting comprises an assay selected from the group consisting of an immunoprecipitation assay, an ELISA, a radioimmunoassay, a Western blot assay and a flow cytometry assay.

98. A method in accordance with Aspect 91, wherein the detecting comprises a flow cytometry assay.

99. A method in accordance with Aspect 89, wherein the at least one primary probe comprises a label, and the detecting presence, absence or quantity of a complex the comprises quantifying the label.

100. A method in accordance with Aspect 99, wherein the label is selected from the group consisting of an enzyme, a radioisotope, a fluorogen, a fluorophore, a chromogen and a chromophore.

101. A method in accordance with Aspect 100, wherein the enzyme is selected from the group consisting of a peroxidase, a phosphatase, a galactosidase and a luciferase.

102. A method in accordance with Aspect 100, wherein the radioisotope is selected from the group consisting of a ³²P, a ³³P, ³⁵S, a ¹⁴C, an ¹²⁵I, an ¹³¹I and a ³H.

103. A method in accordance with Aspect 99, wherein the label is selected from the group consisting of a biotin, a digoxygenin, and a peptide comprising an epitope.

104. A method in accordance with Aspect 69, wherein the detecting presence, absence or quantity of a complex comprises:

contacting the complex with at least one secondary probe that binds the at least one primary probe; and

quantifying the at least one secondary probe.

105. A method in accordance with Aspect 104, wherein the quantifying comprises an assay selected from the group consisting of an immunoprecipitation assay, an ELISA, a radioimmunoassay, a Western blot assay and a flow cytometry assay.

106. A method in accordance with Aspect 104, further comprising selecting or modifying a treatment on the basis of the detection of antibody against XMRV in the sample.

107. A method in accordance with Aspect 106, wherein if antibody against XMRV is detected in the sample, the treatment comprises administrating to the subject a therapeutically effective amount of an anti-viral compound.

108. A method in accordance with Aspect 107, wherein the anti-viral compound is selected from the group consisting of acyclovir, penciclovir (famciclovir), gancyclovir (ganciclovir), deoxyguanosine, foscarnet, idoxuridine, trifluorothymidine, vidarabine, sorivudine, zidovudine, didanosine, zalcitabine, lamivudine, stavudine, abacavir, multinucleoside resistance A, multinucleoside resistance B, nevirapine, delavirdine, efavirenz, adefovir dipivoxil, indinavir, ritonavir, saquinavir, nelfinavir, amprenavir, deoxycytosine triphosphate, lamivudine triphosphate, emticitabine triphosphate, adefovir diphosphate, penciclovir triphosphate, lobucavir triphosphate, amantadine, rimantadine, zanamivir and oseltamivir.

What is claimed is:

1. A method of detecting presence, absence or quantity of antibody against XMRV in a subject, the method comprising:
providing a biological sample comprising antibody from the subject;
forming a mixture comprising a) at least one gammaretrovirus antigen, b) the sample, and c) a competitive probe against the at least one Gammaretrovirus antigen, under conditions sufficient for formation of a complex comprising the at least one Gammaretrovirus antigen and the competitive probe; and
detecting quantity of a complex comprising the at least one Gammaretrovirus antigen and the competitive probe, whereby if the sample comprises antibody against the Gammaretrovirus antigen, the quantity of complex is less than that of a complex formed from a mixture comprising a) the at least one Gammaretrovirus antigen, b) a control sample not comprising antibody against the at least one Gammaretrovirus antigen, and c) the competitive probe against the at least one Gammaretrovirus antigen.

2. A method in accordance with claim 1, wherein the at least one Gammaretrovirus antigen is other than an XMRV gag polypeptide.

3. A method in accordance with claim 2, wherein the XMRV gag polypeptide is selected from the group consisting of a p10 polypeptide, a p15 polypeptide and a p30 polypeptide.

4. A method in accordance with claim 3, wherein the XMRV gag polypeptide is a p30 polypeptide.

5. A method in accordance with claim 1, wherein the method is other than a double antigen sandwich assay.

6. A method in accordance with claim 1, wherein the subject is a human.

7. A method in accordance with claim 1, wherein the subject is a person having, suspected of having, or at risk for developing an XMRV-related disease.

8. A method in accordance with claim 7, wherein the XMRV-related disease is an XMRV-related prostate cancer.

9. A method in accordance with claim 7, wherein the XMRV-related disease is an XMRV-related lymphoma.

10. A method in accordance with claim 9, wherein the XMRV-related lymphoma is selected from the group consisting of an XMRV-related Mantle Cell Lymphoma (MCL) and an XMRV-related Chronic Lymphocytic Leukemia lymphoma (CLL).

11. A method in accordance with claim 6, wherein the XMRV-related disease is an XMRV-related neuroimmune disease.

12. A method in accordance with claim 11, wherein the XMRV-related neuroimmune disease is selected from the group consisting of chronic fatigue syndrome (CFS), Niemann-Pick Type C Disease, fibromyalgia, Multiple Sclerosis (MS), Parkinson's Disease, Amyotrophic Lateral Sclerosis (ALS) and autism.

13. A method in accordance with claim 11, wherein the XMRV-related neuroimmune disease is chronic fatigue syndrome (CFS).

14. A method in accordance with claim 12, wherein the Multiple Sclerosis is Atypical Multiple Sclerosis.

15. A method in accordance with claim 6, wherein the subject exhibits signs and/or symptoms of a neuroimmune disease and/or a lymphoma.

16. A method in accordance with claim 1, wherein the sample is selected from the group consisting of a blood sample, a serum sample, a plasma sample, a sputum sample and a cerebrospinal fluid sample.

17. A method in accordance with claim 1, wherein the sample is selected from the group consisting of a blood sample, a serum sample, a plasma sample and a cerebrospinal fluid sample.

18. A method in accordance with claim 1, wherein the sample is selected from the group consisting of a blood sample, a serum sample and a plasma sample.

19. A method in accordance with claim 18, wherein the sample is a plasma sample.

20. A method in accordance with claim 18, wherein the blood sample is a peripheral blood sample.

21. A method in accordance with claim 1, wherein the at least one Gammaretrovirus antigen comprises a contiguous sequence of at least 4 amino acids of an XMRV polypeptide.

22. A method in accordance with claim 1, wherein the at least one Gammaretrovirus antigen is comprised by at least one cell ex vivo.

23. A method in accordance with claim 22, wherein the at least one cell ex vivo is a mammalian cell expressing at least one Gammaretrovirus antigen ex vivo.

24. A method in accordance with claim 22, wherein the at least one Gammaretrovirus antigen is a polypeptide having at least 95% sequence identity with an SFFV Env protein.

25. A method in accordance with claim 22, wherein the at least one Gammaretrovirus antigen is a polypeptide having at least 96% sequence identity with an SFFV Env protein.

26. A method in accordance with claim 22, wherein the at least one Gammaretrovirus antigen is a polypeptide having at least 97% sequence identity with an SFFV Env protein.

27. A method in accordance with claim 22, wherein the at least one Gammaretrovirus antigen is a polypeptide having at least 98% sequence identity with an SFFV Env protein.

28. A method in accordance with claim 22, wherein the at least one Gammaretrovirus antigen is a polypeptide having at least 99% sequence identity with an SFFV Env protein.

29. A method in accordance with claim 22, wherein the at least one Gammaretrovirus antigen is a polypeptide having 100% sequence identity with an SFFV Env protein.

30. A method in accordance with claim 23, wherein the mammalian cell is a BaF3ER cell.

31. A method in accordance with claim 23, wherein the mammalian cell expressing at least one Gammaretrovirus antigen is a BaF3ER-SFFVEnV cell expressing SFFV Env protein.

32. A method in accordance with claim 1, wherein the at least one Gammaretrovirus antigen is an SFFV antigen.

33. A method in accordance with claim 32, wherein the SFFV antigen is an SFFV Env antigen.

34. A method in accordance with claim 1, wherein the detecting presence, absence or quantity of a complex comprising the at least one Gammaretrovirus antigen and antibody against the at least one Gammaretrovirus antigen comprises contacting the mixture with at least one primary probe directed against antibody from the subject.

35. A method in accordance with claim 34, wherein the at least one primary probe is selected from the group consisting of an antibody, an antigen-binding fragment of an antibody, an aptamer, and an avimer.

36. A method in accordance with claim 34, wherein the at least one primary probe is an antibody or an antigen-binding fragment thereof.

37. A method in accordance with claim 36, wherein the antibody is a polyclonal antibody or a monoclonal antibody.

38. A method in accordance with claim 36, wherein the antigen-binding fragment is an Fab fragment.

39. A method in accordance with claim 1, wherein the competitive probe is an antibody against a gammaretrovirus antigen.

40. A method in accordance with claim 39, wherein the antibody against a gammaretrovirus antigen is a polyclonal antibody.

41. A method in accordance with claim 39, wherein the antibody against a gammaretrovirus antigen is a monoclonal antibody.

42. A method in accordance with claim 34, wherein the antibody against a gammaretrovirus antigen is an antibody against a murine leukemia-related retrovirus.

43. A method in accordance with claim 34, wherein the antibody against a gammaretrovirus antigen is an antibody against a Xenotropic murine leukemia virus.

44. A method in accordance with claim 34, wherein the antibody against a gammaretrovirus antigen is an antibody against a nonecotropic murine leukemia virus.

45. A method in accordance with claim 34, wherein the antibody against a gammaretrovirus antigen is an antibody against a polytropic murine leukemia virus (Mmpv).

46. A method in accordance with claim 34, wherein the antibody against a gammaretrovirus antigen is an antibody against a modified polytropic murine leukemia virus.

47. A method in accordance with claim 34, wherein the antibody against a gammaretrovirus antigen is an antibody against an XMRV.

48. A method in accordance with claim 34, wherein the antibody against a gammaretrovirus antigen is an antibody against an SFFV.

49. A method in accordance with claim 34, wherein the antibody against a gammaretrovirus antigen is selected from the group consisting of an antibody against a gammaretrovirus gag protein, an antibody against a gammaretrovirus env protein and an antibody against a gammaretrovirus pol protein.

50. A method in accordance with claim 34, wherein the antibody against a gammaretrovirus antigen is selected from the group consisting of an antibody against a gammaretrovirus env protein and an antibody against a gammaretrovirus pol protein.

51. A method in accordance with claim 34, wherein the antibody against a gammaretrovirus antigen is an antibody against a gammaretrovirus env protein.

52. A method in accordance with claim 34, wherein the antibody against a gammaretrovirus antigen is an anti gp 55 Env antibody.

53. A method in accordance with claim 51, wherein the antibody against a gammaretrovirus antigen is monoclonal antibody MAb 7C10.

54. A method in accordance with claim 36, wherein the antibody is a polyclonal antibody against at least one Gammaretrovirus antigen.

55. A method in accordance with claim 36, wherein the detecting comprises an assay selected from the group consisting of an immunoprecipitation assay, an ELISA, a radioimmunoassay, a Western blot assay and a flow cytometry assay.

56. A method in accordance with claim 36, wherein the detecting comprises a flow cytometry assay.

57. A method in accordance with claim 34, wherein the at least one primary probe comprises a label, and the detecting presence, absence or quantity of the complex comprises quantifying the label.

58. A method in accordance with claim 57, wherein the label is selected from the group consisting of an enzyme, a radioisotope, a fluorogen, a fluorophore, a chromogen and a chromophore.

59. A method in accordance with claim 58, wherein the enzyme is selected from the group consisting of a peroxidase, a phosphatase, a galactosidase and a luciferase.

60. A method in accordance with claim 58, wherein the radioisotope is selected from the group consisting of a ³²P, a ³³P, ³⁵S, a ¹⁴C, an ¹²⁵I, an ¹³¹I and a ³H.

61. A method in accordance with claim 58, wherein the fluorophore is selected from the group consisting of a fluorescein, a rhodamine, an Alexa Fluor®, a coumarin, an indocyanine or a quantum dot a phycoerythrin, and a green fluorescent protein.

62. A method in accordance with claim 57, wherein the label is selected from the group consisting of a biotin, a digoxygenin, and a peptide comprising an epitope.

63. A method in accordance with claim 1, wherein the detecting presence, absence or quantity of a complex comprises:
contacting the complex with at least one secondary probe that binds the at least one primary probe; and
quantifying the at least one secondary probe bound to the complex, whereby if the sample comprises antibody against the Gammaretrovirus antigen, the quantity of the at least one secondary probe bound to the complex is less than the quantity of the at least one secondary probe bound to a complex formed from a mixture comprising a) the at least one Gammaretrovirus antigen, b) a control sample not comprising antibody against the at least one Gammaretrovirus antigen, and c) the competitive probe against the at least one Gammaretrovirus antigen.

64. A method in accordance with claim 63, wherein the quantifying the at least one secondary probe comprises an assay selected from the group consisting of an immunoprecipitation assay, an ELISA, a radioimmunoassay, a Western blot assay and a flow cytometry assay.

65. A method in accordance with claim 63, wherein the quantifying the at least one secondary probe comprises a flow cytometry assay.

66. A method in accordance with claim 1, further comprising selecting or modifying a treatment on the basis of the detection of antibody against XMRV in the sample.

67. A method in accordance with claim 66, wherein if antibody against XMRV is detected in the sample, the treatment comprises administrating to the subject a therapeutically effective amount of an anti-viral compound.

68. A method in accordance with claim 67, wherein the anti-viral compound is selected from the group consisting of acyclovir, penciclovir (famciclovir), gancyclovir (ganciclovir), deoxyguanosine, foscarnet, idoxuridine, trifluorothymidine, vidarabine, sorivudine, zidovudine, didanosine, zalcitabine, lamivudine, stavudine, abacavir, multinucleoside resistance A, multinucleoside resistance B, nevirapine, delavirdine, efavirenz, adefovir dipivoxil, indinavir, ritonavir, saquinavir, nelfinavir, amprenavir, deoxycytosine triphosphate, lamivudine triphosphate, emticitabine triphosphate, adefovir diphosphate, penciclovir triphosphate, lobucavir triphosphate, amantadine, rimantadine, zanamivir and oseltamivir.

69. A method of detecting, diagnosing, monitoring or managing an XMRV-related disease in a subject, the method comprising:
providing a biological sample comprising antibody from the subject;
forming a mixture comprising a) at least one Gammaretrovirus antigen and b) the sample, under conditions sufficient for formation of a complex comprising the at least one Gammaretrovirus antigen and antibody against the at least one Gammaretrovirus antigen if present in the antibody from the subject; and
detecting presence, absence or quantity of a complex comprising the at least one Gammaretrovirus antigen and antibody against the at least one Gammaretrovirus antigen.

70. A method in accordance with claim 69, wherein the at least one Gammaretrovirus antigen is other than a gammaretrovirus gagp30.

71. A method in accordance with claim 69, wherein the method is other than a double antigen sandwich assay.

72. A method in accordance with claim 69, wherein the subject is a person having, suspected of having, or at risk for developing an XMRV-related disease.

73. A method in accordance with claim 72, wherein the XMRV-related disease is an XMRV-related prostate cancer.

74. A method in accordance with claim 72, wherein the XMRV-related disease is an XMRV-related lymphoma.

75. A method in accordance with claim 74, wherein the XMRV-related lymphoma is selected from the group consisting of a XMRV-related Mantle Cell Lymphoma (MCL) and an XMRV-related Chronic Lymphocytic Leukemia lymphoma (CLL).

76. A method in accordance with claim 72, wherein the XMRV-related disease is an XMRV-related neuroimmune disease.

77. A method in accordance with claim 76, wherein the XMRV-related neuroimmune disease is selected from the group consisting of chronic fatigue syndrome (CFS), Niemann-Pick Type C Disease, fibromyalgia, Multiple Sclerosis (MS), Parkinson's Disease, Amyotrophic Lateral Sclerosis (ALS) and autism.

78. A method in accordance with claim 77, wherein the Multiple Sclerosis is Atypical Multiple Sclerosis.

79. A method in accordance with claim 72, wherein the subject exhibits signs and/or symptoms of a neuroimmune disease and/or a lymphoma.

80. A method in accordance with claim 69, wherein the sample is selected from the group consisting of a blood sample, a serum sample, a plasma sample, and a cerebrospinal fluid sample.

81. A method in accordance with claim 61, wherein the blood sample is a peripheral blood sample.

82. A method in accordance with claim 69, wherein the at least one Gammaretrovirus antigen comprises a contiguous sequence of at least 4 amino acids of an XMRV polypeptide.

83. A method in accordance with claim 69, wherein the at least one Gammaretrovirus antigen is comprised by at least one cell ex vivo.

84. A method in accordance with claim 83, wherein the at least one cell ex vivo is a mammalian cell expressing at least one Gammaretrovirus antigen ex vivo.

85. A method in accordance with claim 84, wherein the mammalian cell expressing the at least one gammaretrovirus antigen is a BaF3ER cell.

86. A method in accordance with claim 84, wherein the mammalian cell expressing at least one Gammaretrovirus antigen is a BaF3ER-SFFVEnV cell expressing SFFV Env protein.

87. A method in accordance with claim 69, wherein the at least one Gammaretrovirus antigen is an SFFV antigen.

88. A method in accordance with claim 87, wherein the SFFV antigen is an SFFV Env antigen.

89. A method in accordance with claim 69, wherein the detecting presence, absence or quantity of a complex comprising the at least one Gammaretrovirus antigen and antibody against the at least one Gammaretrovirus antigen comprises contacting the mixture with at least one primary probe directed against antibody from the subject.

90. A method in accordance with claim 89, wherein the at least one primary probe is selected from the group consisting of an antibody, an antigen-binding fragment of an antibody, an aptamer, and an avimer.

91. A method in accordance with claim 89, wherein the at least one primary probe is an antibody or an antigen-binding fragment thereof.

92. A method in accordance with claim 91, wherein the antibody is a polyclonal antibody or a monoclonal antibody.

93. A method in accordance with claim 91, wherein the antigen-binding fragment is an Fab fragment.

94. A method in accordance with claim 91, wherein the antibody is an anti gp 55 Env antibody.

95. A method in accordance with claim 91, wherein the antibody is a monoclonal antibody MAb 7C10.

96. A method in accordance with claim 91, wherein the antibody is a polyclonal antibody against at least one Gammaretrovirus antigen.

97. A method in accordance with claim 91, wherein the detecting comprises an assay selected from the group consisting of an immunoprecipitation assay, an ELISA, a radioimmunoassay, a Western blot assay and a flow cytometry assay.

98. A method in accordance with claim 91, wherein the detecting comprises a flow cytometry assay.

99. A method in accordance with claim 89, wherein the at least one primary probe comprises a label, and the detecting presence, absence or quantity of a complex the comprises quantifying the label.

100. A method in accordance with claim 99, wherein the label is selected from the group consisting of an enzyme, a radioisotope, a fluorogen, a fluorophore, a chromogen and a chromophore.

101. A method in accordance with claim 100, wherein the enzyme is selected from the group consisting of a peroxidase, a phosphatase, a galactosidase and a luciferase.

102. A method in accordance with claim 100, wherein the radioisotope is selected from the group consisting of a ³²P, a ³³P, ³⁵S, a ¹⁴C, an ¹²⁵I, an ¹³¹I and a ³H.

103. A method in accordance with claim 99, wherein the label is selected from the group consisting of a biotin, a digoxygenin, and a peptide comprising an epitope.

104. A method in accordance with claim 69, wherein the detecting presence, absence or quantity of a complex comprises:
contacting the complex with at least one secondary probe that binds the at least one primary probe; and
quantifying the at least one secondary probe.

105. A method in accordance with claim 104, wherein the quantifying comprises an assay selected from the group consisting of an immunoprecipitation assay, an ELISA, a radioimmunoassay, a Western blot assay and a flow cytometry assay.

106. A method in accordance with claim 104, further comprising selecting or modifying a treatment on the basis of the detection of antibody against XMRV in the sample.

107. A method in accordance with claim 106, wherein if antibody against XMRV is detected in the sample, the treatment comprises administrating to the subject a therapeutically effective amount of an anti-viral compound.

108. A method in accordance with claim 107, wherein the anti-viral compound is selected from the group consisting of acyclovir, penciclovir (famciclovir), gancyclovir (ganciclovir), deoxyguanosine, foscarnet, idoxuridine, trifluorothymidine, vidarabine, sorivudine, zidovudine, didanosine, zalcitabine, lamivudine, stavudine, abacavir, multinucleoside resistance A, multinucleoside resistance B, nevirapine, delavirdine, efavirenz, adefovir dipivoxil, indinavir, ritonavir, saquinavir, nelfinavir, amprenavir, deoxycytosine triphosphate, lamivudine triphosphate, emticitabine triphosphate, adefovir diphosphate, penciclovir triphosphate, lobucavir triphosphate, amantadine, rimantadine, zanamivir and oseltamivir.
